(19) United States
(12) Patent Application Publication
(10) Pub. No.: US 2026/0341430 A1
(43) Pub. Date: Oct. 29, 2026

(54) DATASTORE AGNOSTIC USER ADMINISTRATION SYSTEM

(71) Applicant: Oracle International Corporation, Redwood Shores, CA (US)

(72) Inventors: Justin Page, Bountiful, UT (US); Andrei Vorobiev, Henderson, NV (US); Joel Rowles, Briar, WA (US)

(73) Assignee: Oracle International Corporation, Redwood Shores, CA (US)

(21) Appl. No.: 19/192,030

(22) Filed: Apr. 28, 2025

(51) Int. Cl. G16H 10/60; G06Q 10/1093

(52) U.S. Cl. CPC G16H 10/60; G06Q 10/1093

(57) ABSTRACT

The present disclosure relates to techniques for managing service requests in a unified synchronization system that enables data consistency and synchronization across a multi-entity, distributed environment comprising various service providers and associated datastores. The disclosed techniques may involve a schema-agnostic datastore configured to store aggregated data from diverse datastores via a set of decoupled connectors. The decoupled connectors may facilitate data retrieval and standardization by using one or more machine-learning techniques including encoding, cross-terminology mapping, and duplicate data detection. Additionally, the disclosed techniques may facilitate service requests to be processed in real-time, with execution updates propagated across all connected systems via a set of event streams. The disclosed techniques may further enhance data interoperability, administrative efficiency, and user experience by providing a scalable, unified system for managing registration and scheduling processes across disparate datastores.

### BACKGROUND

Managing patients'journeys efficiently and effectively is a growing concern for hospitals, clinics, and healthcare providers, as avoidable delays in patients'journeys result in frustrating and stressful experience for patients. Consequently, smart and seamless scheduling of patients, seamless registration at front desks, and patient centric customized access to healthcare services is needed. In comparison, existing electronic health records (EHR) ecosystems are fragmented and are operating in silos, requiring patients to register separately at each hospital or healthcare facility they visit, leading to redundant data entry, inconsistencies in data; as a result, it becomes a challenge to maintain reliable, accurate, and consistent medical records across various healthcare providers. Existing patient registration and scheduling systems are not capable of managing complexities of multi-institutional healthcare environments, as patients may frequently seek care from multiple hospitals, specialty clinics, and healthcare networks. In these scenarios, providers may not have access to a patient's prior medical records from other healthcare providers, making it difficult for providers to make informed evidence based decision-making related to diagnoses of disease and relevant treatment management. The fragmented healthcare records may also add delays to providing timely care to patients, increase administrative overheads, and pose significant health risks—misdiagnoses and mistreatments—to patients because of incomplete medical records and history. Additionally, healthcare providers often use local EHRs that may not be interoperable with other EHRs; as a result, data is manually entered that may be received through faxes, leading to incomplete (or sometimes redundant) investigations, resulting in misdiagnosis and mistreatment errors and higher costs that may result in dissatisfied patients.

Moreover, when patients visit new hospitals, administrators may have to manually verify prior records, cross-check insurance details, and collect medical history, increasing wait times of patient and workload for administrative assistants. Moreover, seamless referral and scheduling across different healthcare facilities becomes a challenge, as hospitals typically operate independent scheduling systems that are supported by their EHRs, making it difficult for patients to seamlessly schedule, reschedule, or transfer appointments across different healthcare networks. Without a unified system, overlapping appointments, double bookings, and scheduling conflicts may frequently arise, leading to care and operational inefficiencies.

Fragmented data governance across multiple healthcare institutions may also contribute to data inconsistencies, manual reconciliation efforts, and potential errors in medical records. Therefore, advanced, globally integrated patient administration systems are needed to enable seamless patient registration, centralized appointment scheduling, automated record synchronization, and efficient cross-hospital data exchange.

### SUMMARY

Some aspects of the present disclosure relate to techniques for managing service requests in a unified system that enables data consistency and synchronization across a multi-entity, distributed data mesh comprising a plurality of service providers and associated datastores. The disclosed techniques may include a schema-agnostic datastore configured to store schema-agnostic data, retrieved from a plurality of datastores in source-specific formats, in a schema-agnostic format. Additionally, the disclosed techniques may facilitate service requests to be processed in real-time, with execution updates propagated and synchronized across all service providers within the distributed data mesh.

The disclosed techniques may involve receiving a request via a user interface (UI) from a user to avail a service from one or more service providers of the plurality of service providers. The request may include a service scheduling request (e.g., an appointment scheduling request with a healthcare provider) or a registration request for registering with the one or more service providers (healthcare providers or clinics). Once the request is received by a service provider, schema-agnostic data that may include users'information may be accessed from a schema-agnostic datastore. The users'information may be generated via a set of decoupled, bidirectional connectors which may be configured to retrieve users'information from the plurality of datastores within the distributed data mesh. The retrieved users'information which may be in the source-specific format (structured according to distinct terminologies, schemas, and conventions used by various datastores) may be transformed and standardized into a schema-agnostic format using one or more machine-learning techniques before storing in the schema-agnostic datastore.

According to the disclosed techniques, the decoupled connectors may facilitate data normalization by employing one or more encoding techniques to standardize the retrieved users'and service providers'information. For instance, in a clinical setting, patient records from different hospitals may use varying formats to store numerical values, such as blood pressure readings recorded as “120/80 mmHg” in one system and “BP: 120 systolic, 80 diastolic” in another. To standardize such numerical data, z-score normalization may be applied, transforming values into a common scale that may account for statistical distribution. Similarly, categorical data, such as diagnostic codes or medication names, may be processed using one-hot encoding or label encoding, allowing machine-learning models to interpret them efficiently and effectively. Additionally, word embeddings such as Word2Vec and GloVe may be used to convert clinical text (e.g., physician notes, discharge summaries) into dense vector representations, capturing semantic relationships between various medical terms.

Next, the normalized data may undergo terminology mapping to maintain consistency across diverse terminologies, used by different service providers and datastores (e.g., medical databases or electronic health records (EHRs)). According to some aspects, terminology mapping may be fully automated using AI models trained on historical mappings across systems. In other aspects, the terminology mapping may involve manual verification. Ontology-based mappings (e.g., using SNOMED CT or BERTMap) may also be used, particularly in the clinical domain where semantic alignment is critical.

For example, one EHR system may record a condition as “myocardial infarction,” while in another EHR it may use “heart attack” or ICD-10 code “I21.9” to describe the same condition. To cater for these variations, transformer-based language models such as BERT, BioBERT, or SciBERT may be used to identify or extract terminologies from the normalized data, identifying equivalent terms even when they may be phrased differently by different providers. The extracted terms may then be mapped to predefined, standardized ontologies—such as SNOMED CT for clinical concepts, RxNorm for medications, or LOINC for lab results—using knowledge graph embeddings like TransE, ComplEx, or RotatE. By applying these techniques, transformed data may be generated, enabling seamless interoperability between disparate datastores and supporting accurate, real-time decision-making within the multi-entity, distributed data mesh.

The transformed data associated with each datastore of the plurality of datastores may be aggregated to generate a schema-agnostic data that may then be stored into the schema-agnostic datastore. To further enhance accuracy of data and prevent duplication, various record linkage models may be utilized to detect duplicate records associated with the same user across multiple datastore. For instance, a patient visiting multiple hospitals may have separate records but with slightly different names. Additionally, variations in demographic data, such as differences in date of birth formats or little misspellings in addresses, can make it difficult to match various records of the same person accurately.

According to the disclosed techniques, the record linkage models such as support vector machines (SVM), pairwise logistic regression, expectation-maximization (EM) algorithms, or Siamese networks may be used to analyze similarities within the schema-agnostic data based on multiple attributes. For example, a pairwise logistic regression model may compare attributes like name, address, phone number, and medical history, assigning a probability score indicating a likelihood that two records belong to the same person. Similarly, a Siamese network model may process textual and numerical inputs to determine whether records with variations in spelling, formatting, or missing fields may correspond to the same user.

Once duplicate records are detected, the system may generate a merged data or a merged user profile, consolidating medical history, previous diagnoses, prescriptions, and lab results into a single unified record. The transformation process may allow the system to convert heterogeneous data from the plurality of datastores into a unified format, making it accessible in real-time for executing service requests. For instance, while scheduling patients'appointments, this may enable accurate patient identification, allowing availability checks and registrations to be based on a comprehensive, up-to-date patient's profile that is not fragmented and stored across the different datastores.

Once a unified user profile is created, the system may identify one or more service providers of the plurality of service providers that may be relevant to the user depending on the user's request. The decoupled connectors may then retrieve resource metrics associated with the service provider, from the plurality of datastores associated with the one or more service providers. The resource metrics may include availability of the provider, availability of the provider's time slot, and availability of relevant resource. For example, in a clinical setting, the resource metrics may correspond to a physician's availability, operating room occupancy, the number of available hospital beds, or the availability of specialized medical equipment such as MRI machines. The resource metrics may be displayed to the user via the UI, allowing them to select a preferred service provider and the time slot. According to some aspects of the disclosure, the user may be an authorized front-office administrator of a service provider (e.g., a hospital receptionist, physician assistant, or a scheduling coordinator who may be responsible for managing patients'registrations and appointment scheduling workflows). In some other aspects, the user may be a person (e.g., a patient scheduling a medical consultation) who may be directly communicating with the system to request a scheduling of a service from a service provider or a registration request for registering with one or more service providers for healthcare services.

Upon receiving the user's preference, the system may schedule the requested service with the selected service provider and update the schema-agnostic datastore accordingly. To facilitate real-time synchronization of service request updates, the disclosed techniques may use an event stream which may be a transactional event queue (TEQ). The TEQ may include an event listener that may detect updates related to service requests, registrations, or resource availability within the schema-agnostic datastore and the plurality of datastores within the distributed data mesh. Additionally, a message broker may be used to propagate the updates to the one or more service providers, allowing the changes to be reflected across all connected systems (i.e., the service providers associated with the user). This real-time synchronization mechanism may enable the service providers to maintain up-to-date user's information and minimize discrepancies or inconsistencies between systems.

The disclosed techniques may also allow for detecting dynamic updates within a datastore of the plurality of datastores and deploying them efficiently. For example, if a datastore within the distributed data mesh has changed, such as a new registration or an update to an existing scheduled service, the event stream may dynamically capture this change and trigger various synchronization mechanisms. The decoupled connectors may then propagate the update to the schema-agnostic datastore and other datastores to ensure that the most current information is available at all datastores.

In addition to scheduling, the disclosed techniques may support the user's registration across multiple service providers. When the user submits a registration request via the UI, the system may add the registration data to the schema-agnostic datastore and synchronize it with other relevant datastores. This approach may enable centralized registration management while allowing seamless integration with distributed service providers. By leveraging real-time synchronization and schema-agnostic data storage, the system may improve efficiency in handling registrations and scheduling of the user across multiple healthcare entities.

In some embodiments, a system is provided that includes one or more data processors and a non-transitory computer readable storage medium containing instruction which, when executed on the one or more data processors, cause the one or more data processors to perform part or all of one or more methods disclosed herein.

In some embodiments, a computer-program product tangibly embodied in a non-transitory machine-readable storage medium, including instructions configured to cause one or more data processors to perform part or all of one or more methods or processes disclosed herein.

In some embodiments, a system is provided that includes one or more means to perform part or all of one or more methods or processes disclosed herein.

The terms and expressions which have been employed are used as terms of description and not of limitation, and there is no intention in the use of such terms and expressions of excluding any equivalents of the features shown and described or portions thereof, but it is recognized that various modifications are possible within the scope of the invention claimed. Thus, it should be understood that although the present invention as claimed has been specifically disclosed by embodiments and optional features, modification and variation of the concepts herein disclosed may be resorted to by those skilled in the art, and that such modifications and variations are considered to be within the scope of this invention as defined by the appended claims.

### BRIEF DESCRIPTION OF THE DRAWINGS

The present disclosure is described in conjunction with the appended figures:

FIG. 1 illustrates an exemplary block diagram of a unified synchronization system housed in a cloud network in accordance with some aspects of the present disclosure.

FIG. 2 illustrates an overview diagram of a connector configured to standardize information from multiple datastores into a schema-agnostic format in accordance with some aspects of the present disclosure.

FIG. 3 illustrates an overview diagram of a duplicate detection model to detect user information associated with the same user from the schema-agnostic data according to some aspects of the present disclosure.

FIG. 4 illustrates a multi-tenancy structure of the unified synchronization system in accordance with some aspects of the present disclosure.

FIG. 5 shows a sequence diagram for processing a service request received from a user via an API gateway according to some aspects of the present disclosure.

FIG. 6A illustrates an exemplary user interface (UI) of an authorized user showing a user's administration dashboard according to some aspects of the present disclosure.

FIG. 6B illustrates an exemplary UI showing a user's information of a user associated with multiple service providers according to some aspects of the present disclosure.

FIG. 7 shows an example flowchart of a system for managing and synchronizing data across multiple datastores according to some aspects of the present disclosure.

FIG. 8 depicts a simplified diagram of a distributed system for implementing an embodiment of the present disclosure.

FIG. 9 is a simplified block diagram of a cloud system environment in accordance with certain aspects of various embodiments of the invention.

FIG. 10 illustrates an exemplary computer system that may be used to implement certain aspects of the present disclosure.

### DETAILED DESCRIPTION

Some embodiments of the present disclosure relate to a unified synchronization system for managing service requests within a multi-entity system comprising various service providers and associated datastores by leveraging a schema-agnostic datastore. The unified synchronization system may utilize a set of decoupled connectors for normalization of the retrieved from various datastores, associated with different service providers, into a unified representation. Moreover, the unified synchronization system may enable real-time data execution of services (i.e., scheduling services or registration of new users) while allowing updates associated with the executed services to be synchronized across the service providers via a set of event streams and the decoupled connectors.

According to some aspects, the unified synchronization system may operate as part of a distributed data mesh comprising various datastores and associated service providers. The service providers (also referred herein as providers) may integrate or leverage the unified synchronization system alongside their existing internal control systems. This may allow the service providers to manage their internal workflows separately while also being able to manage service requests at a centralized, connected level by leveraging the unified synchronization system.

By maintaining a schema-agnostic data store, the unified synchronization system may enable seamless aggregation and standardization of information that comprises a user's information and a provider's information from multiple (external) datastores into the unified representation or format, thereby synchronizing updates that are made by any entity within the connected data mesh (i.e., the unified synchronization system itself, the datastores associated with the service providers, or third-party service providers) to be consistently synchronized and the synchronization updates are reflected reliably across all relevant datastores. This approach may enhance data accuracy, reduce data fragmentation, and improve operational efficiency by enabling the service providers to have access to up-to-date and synchronized data.

According to disclosed techniques, the unified synchronization system may not directly integrate with the datastores, instead it may utilize the set of decoupled connectors to handle data ingestion and synchronization between the schema-agnostic datastore and the (external) datastores. According to some aspects, the decoupled connectors may enable bidirectional data exchange without requiring the unified synchronization system to be tightly coupled with any specific datastore. Additionally, the decoupled connectors may work on an event-driven architecture, i.e., when an update occurs in the unified synchronization system (e.g., in a healthcare setting, regarding a patient's registration or appointment scheduling), the change may be sent via the event stream, as an event to a message queue (e.g., a transactional event queue in Oracle™). Alternatively, any update within the datastores (e.g., update to a patient's data), may be detected or captured via the event stream associated with the particular datastore, and may be synchronized with the schema-agnostic datastore.

To maintain compatibility with the diverse datastores, the decoupled connectors may be configured to convert the data being sent from the schema-agnostic datastore back to the datastores in the specific format or the schema as required by each datastore. This may be implemented using an outbound adapter within the decoupled connector. The outbound adapter may include a schema mapper and an autoencoder that may transform the schema-agnostic data into the appropriate source-specific format before synchronization. This transformation may allow the system to accommodate heterogeneous data formats across different service providers, enabling consistency and interoperability while maintaining the flexibility of having the schema-agnostic datastore. Each datastore within the data mesh may be assigned a separate decoupled connector, so that each connector may handle schema-mapping (schema-conversion), validations and error resolutions specific to a particular datastore.

In the present disclosure, the data mesh refers to an interconnected, multi-entity system that may include the unified synchronization system, control systems (that may handle broader service provider operations including revenue cycle management and compliance tracking), one or more datastores (e.g., EHR systems including Millennium, Allscripts, i.s.h.med etc.) associated with the service providers. The data mesh may also include other datastores and control systems associated with other service providers integrating with the unified synchronization system.

The control systems may refer to the systems that service providers may use to manage and oversee administrative, financial, and operational workflows. These systems may be deployed on-premises or in cloud-based environments, providing flexibility based on the specific needs of the service provider. In a healthcare setting, the control system may be responsible for handling a wide range of services including the patients'registration, billing, claims processing, compliance management, and overall revenue cycle operations. According to some aspects, the control system offered by Oracle™ may include an integrated revenue cycle suite (IRCS), health information management (HIM), oracle health patient accounting (OHPACT), and common financial clearance (CFC).

According to some aspects of the disclosure, the unified synchronization system may be the oracle health patient administration service (OHPAS™), which is a front-office system for patient administration operations of a healthcare practice offered by Oracle™. The system is built on a modular architecture that leverages a set of well-defined application programming interfaces (APIs) to facilitate seamless integration with existing healthcare ecosystems. OHPAS™ utilizes Millennium and v500 databases, enabling interoperability and data consistency across various healthcare providers.

Furthermore, users accessing the unified synchronization system may encompass individuals intending to request services, register with new service providers, or manage existing service engagements. The users may include customers, clients, patients, or employees interacting with various service domains, such as healthcare, finance, education, logistics, or telecommunications. Additionally, administrative personnel of the service providers may also access the system, including front-office administrators, coordinators, or service managers responsible for handling registration, scheduling, resource allocation, and other operational workflows based on real-time service availability and users'requirements.

According to some aspects of the disclosure, the decoupled connectors may include various processes to standardize data accessed from the datastores into a schema-agnostic, structured format to be stored in the schema-agnostic datastore. According to some aspects, the structured format may be similar to the HL7 (health level 7), a set of international standards (or standard data format) that is already being used for exchanging healthcare information (e.g., like ADT-admin/discharge/transfer data) between various healthcare providers. In some other aspects, the structured format may be similar to the FHIR (fast healthcare interoperability resources), another modern standard used for web-based healthcare data (e.g., patient encounter, observation) exchange using RESTful APIs, JSON or XML files.

The standardization process may include a cross-terminology mapping model, a format standardization model, and a duplicity detection model. The cross-terminology mapping model may leverage one or more machine-learning (ML) models configured to identify and align medical terminologies across the different datastores into a (predefined) standardized format. The ML models may include natural language processing (NLP), ontology-based mappings to recognize variations in medical codes, terminologies, and data structures across disparate healthcare systems.

Additionally, the duplicate detection model may leverage advanced NLP and record-linkage techniques to identify data from disparate datastores, corresponding to the same user. The duplicate detection model may utilize named entity recognition (NER) with transformer-based models like decoupled encoder representations from transformers (BERT), or spaCy (an open-source NLP library optimized for high-performance text processing, including entity recognition, tokenization, and dependency parsing), to extract and standardize one or more relevant fields.

Once potential duplicates are identified, similarity estimation techniques may be used to quantify the degree of relatedness between the duplicate data or duplicate records represented by a similarity score. If the similarity score exceeds a predefined confidence threshold, the system may merge the detected duplicate records into a unified or a merged record (also referred herein as merged data), enabling data integrity and reducing fragmentation across various datastores.

Following the standardization processes, the data may be stored into the schema-agnostic datastore, which may further be accessed by a services unit within the unified synchronization system to manage users'requests. The services unit may support various functions, including scheduling, registration, documentation, and may be accessed by both front-office administrators and subjects through their respective user interfaces. These interactions may occur via an API gateway, enabling seamless access to the services unit.

Once a service request is received form the user, such as scheduling a service with a service provider, the services unit may retrieve relevant user and provider information from the schema-agnostic datastore and evaluate resources availability based on factors like provider capacity, operational constraints, and historical data. For instance, if the request includes booking an appointment, the services unit may assess providers'appointment calendar, availability of specialists, and required equipment or resources for providing the service to the user. Based on these evaluations, a suitable service provider may be selected to execute the requested service. Once the service is scheduled, the schema-agnostic datastore may be updated to store the details of the assigned service including the selected provider, scheduled time, and any relevant contextual information. This update may allow the unified synchronization system to maintain a consistent and up-to-date representation of the scheduled services across all associated entities. Simultaneously, the provider's control system may receive real-time updates via the event stream, enabling the provider to process the requests efficiently, allocate necessary resources for them, and prepare for the service execution.

Additionally, other datastores linked to the user may be synchronized using the decoupled connectors to ensure the newly scheduled service has access to the synchronized datastores. The decoupled connectors facilitate seamless data exchange between the schema-agnostic datastore and distributed datastores, ensuring that all relevant systems—such as those belonging to other service providers that may be associated with the user—are updated accordingly. This synchronization helps maintain consistency across multiple entities, allowing for a better coordination and an accurate record-keeping of the scheduled services.

For example, if a patient submits a request seeking an appointment for a specific treatment, the services unit may analyze the user's information to identify clinics that may be able to provide the specific treatment to the patient. It may then retrieve providers'information associated with the identified clinics from the schema-agnostic datastore and assess resource availability metrics, such as specialists'schedules, required medical equipment, and consultation hours. Once a clinic with necessary resources is identified, an appointment may be scheduled at that clinic. The schema-agnostic datastore may be updated with the confirmed appointment details, and the clinic's control system may receive real-time updates regarding the scheduled appointment via the event stream. Simultaneously, other clinics where the patient may be registered can also be updated to reflect the upcoming appointment, enabling synchronization of records across all linked datastores.

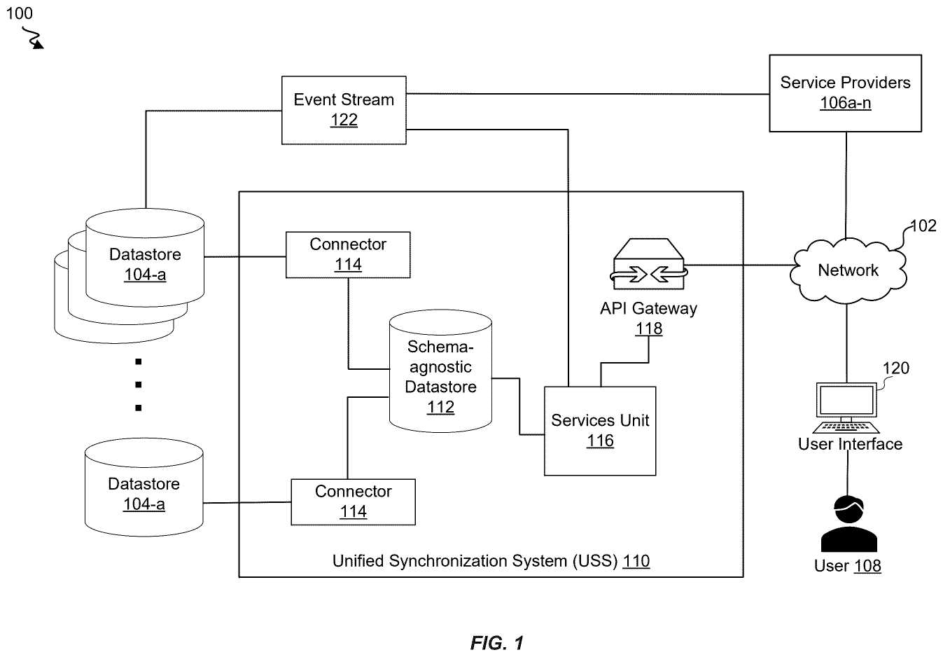

FIG. 1 illustrates an exemplary block diagram of a unified synchronization system 110 at a cloud network in accordance with some aspects of the present disclosure. The system may include a plurality of datastores 104a-n, a unified synchronization system 110, a network 102, a plurality of service providers 106a-n, a user 108, and a user interface (UI) 120. Further, the unified synchronization system 110 may include a schema-agnostic datastore 112, a services unit 116, one or more connectors 114, and an API gateway 118.

The unified synchronization system 110 may serve as a centralized system for managing various processes, where different service providers may access and update data from a schema-agnostic datastore 112. The schema-agnostic datastore 112 may retrieve and normalize data from various distributed datastores 104a-n via the connectors 114, which may perform data normalization and terminology mapping to enable consistency across various heterogeneous systems.

The services unit 116 may provide a suite of functionalities that may allow service providers 106a-n to access and manage data relevant to their operations. The service providers 106a-n may include hospitals, educational institutions, legal firms, or other entities that may require a centralized system for handling processes such as registrations, appointments'scheduling, records'management, or transactional services. The user 108 may be an authorized representative of a service provider (e.g., front office managers, educational staff, lawyers) or an individual availing services (e.g., students, patients, clients). Through the UI 120, connected via the network 102, the user 108 may interact with the services unit 116 to initiate or manage various service requests.

The schema-agnostic datastore 112 may operate as a unified data access layer, retrieving information from multiple datastores 104a-n used by different service providers and storing it in a format that may enable interoperability of various systems. Event streams 122 may be utilized to detect real-time changes occurring in any of the datastores 104a-n or within the schema-agnostic datastore 112, prompting the services unit 116 to synchronize updates across various connected systems. This synchronization may occur bidirectionally, using the connectors 114, enabling modifications or updates originating at one system to propagate to relevant systems without causing any data fragmentation. For example, if a service provider 106 (e.g., an administration office of a university) updates a student's enrollment status in its internal system, or the datastore 104, the change may be detected via an event stream 122 and synchronized across the schema-agnostic datastore 112 along with other connected systems, such as financial aid offices, housing services, or affiliated institutions relying on the same data. Similarly, if a legal firm updates a client's case status, the relevant updates may be propagated to associated legal databases, court scheduling systems, or other authorized entities requiring access to the latest updated information.

Additionally, using the unified, centralized architecture, the unified synchronization system 110 may provide a flexible and scalable approach to managing distributed data environments while maintaining a decoupled structure that supports interoperability across diverse and heterogenous service providers. The connectors 114 and API gateway 118 may enable seamless communication between external systems and the services unit 116, facilitating efficient data access, updates, and synchronization across a cloud-based network.

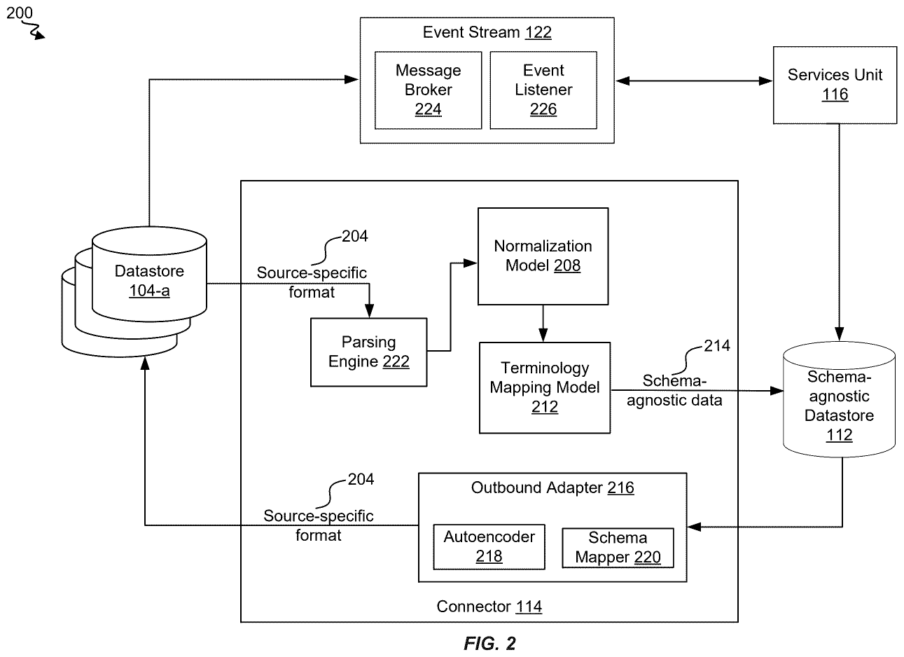

FIG. 2 illustrates an overview diagram of the connector 114 configured to standardize information from multiple datastores 104a-n into a schema-agnostic format. The connectors 114 may include a parsing engine 222, a normalization model 208, a terminology mapping model 212, and an outbound adapter 216. The outbound adapter 216 may include a schema mapper 220, and an autoencoder 218. Additionally, the event stream 206 may include a message broker 224 and an event listener 226. These components, working in tandem, may enable synchronization, facilitating real-time data standardization and bidirectional updates, across the service providers 106a-n within the data mesh.

The information retrieved from the datastores 104a-n, which may exist in various source-specific formats 204, may first be processed by the parsing engine 222. The parsing engine 222 may be configured to extract structured data from various datastores 104a-n, each of which may have stored data in their respective formats, including relational databases, XML, JSON, HL7, EDI, or proprietary file formats. The parsing engine 222 may utilize natural language processing (NLP) models, such as transformer-based models (e.g., BERT, GPT), to extract semantic meaning from the unstructured text data. Additionally, sequence-to-sequence models, recurrent neural networks (RNNs), or rule-based parsers may also be leveraged to structure the extracted data efficiently. For example, in a financial domain, transaction details from bank statements may be parsed and converted into a structured format, while in a healthcare domain, patients'registration data from different hospital systems may be extracted and prepared for standardization.

The output from the parsing engine 222 may be sent to the normalization model 208, that may be configured to standardize attributes across datastores by applying machine-learning techniques such as autoencoders, decision trees, and embedding-based models (e.g., Word2Vec, FastText). The normalization model 208 may be trained on historical datasets comprising a set of variations of entity representations, enabling appropriate transformations. For instance, if different hospital systems store birth dates in varying formats (e.g., YYYY-MM-DD vs. DD/MM/YYYY), the normalization model 208 may standardize these formats into a unified representation. Similarly, in an e-commerce setting, product descriptions from various sellers may be standardized by normalizing attributes like pricing units, category labels, and inventory quantities.

The normalized data may then be processed by the terminology mapping model 212, which may apply ontology-based and machine learning-based mapping techniques to unify terminologies across different sources into a predefined international standard (e.g., HL7 or FHIR format). The terminology mapping model 212 may include knowledge graph embeddings, ontology alignment algorithms, and deep learning models trained on domain-specific vocabularies.

For example, in a healthcare scenario, data retrieved from a hospital's datastore, possibly containing custom or proprietary field names for patient encounters, procedures, or lab results, may be mapped and converted into a common set of standardized. The mapping process may align proprietary codes (e.g., internal diagnosis codes) with standard terminologies like ICD-10 or SNOMED CT, enabling semantic interoperability across systems.

To achieve this, the terminology mapping model 212 may use pre-trained language models (e.g., BioBERT for clinical text) to extract and contextualize relevant terms from the normalized data. The extracted terms may then be mapped to standardized ontology identifiers using graph embedding models (e.g., TransE, ComplEx, or RotatE) or ontology alignment models (e.g., BERTMap, DeepMap). According to some aspects, manual verification or correction may also be needed for managing terminology mappings.

In other domains, such as finance, the terminology mapping model 212 may reconcile differing field names or codes used by various banking APIs to refer to transaction types, account classes, or fee categories. The output of the terminology mapping model 212 may be the schema-agnostic data 214, which is stored in the agnostic datastore 112, enabling different service providers 106a-n to interact with a unified and semantically aligned data

In some aspects, the connectors 114 may be bidirectional, and may enable data exchange not only from multiple datastores 104a-n to the schema-agnostic datastore 112 but also synchronizing data back to the source systems in their respective source-specific formats 204. This may be achieved using the outbound adapter 216, which may include the schema mapper 220 and the autoencoder 218. The schema mapper 220 may transform the schema-agnostic data 214 into the respective schema of the target datastores 104a-n, using techniques such as generative adversarial networks (GANs) or rule-based transformation engines. The autoencoder 218 may further improve these transformations by learning optimal representations for encoding and decoding data. For example, if a standardized patient record stored in the schema-agnostic datastore 112 needs to be updated in a hospital's EHR system, the schema mapper 220 may convert it back into the hospital's proprietary format while preserving semantic integrity of the standardized patient record. Similarly, in a financial setting, if a transaction record may be modified in the schema-agnostic datastore 112, the autoencoder 218 may encode the updated information and write it to the datastore of the respective banking institutions in their required formats.

Furthermore, the schema-agnostic datastore 112 may categorize the schema-agnostic data 214 into activity data and reference data, facilitating efficient synchronization and management across various knowledge domains. The activity data may represent dynamic, operational information that may frequently be updated within a data mesh, such as user transactions, service requests, registrations, or scheduling events. This activity data may originate from a user's interactions and be continuously modified as new events occur. For example, in a financial domain, the activity data may include real-time payment transactions, while in a healthcare setting, it may comprise patient admissions, appointment bookings, or treatment updates.

In contrast, the reference data may consist of relatively static or structured information that provides contextual support for operations but may not frequently be modified. This may include standardized classification codes, institutional identifiers, user credentials, or configuration parameters. For instance, in a healthcare system, reference data may encompass medical coding standards, practitioner credentials, or facility directories, while in a financial domain, it may include currency exchange rates, account types, or regulatory classifications.

According to some aspects, the synchronization process facilitated by the connectors 114 and event stream 122 may enable updates to the activity data within the schema-agnostic datastore 112 to propagate it to relevant datastores 104a-n in real time. The message broker 224 and the event listener 226, used by the event stream 206, may detect changes that may be occurring in any of the datastores 104a-n or within the schema-agnostic datastore 112, and in-turn may trigger the services unit 116 to prompt the relevant datastores to do synchronization of the data. By distinguishing and separately maintaining the activity data and reference data, the system 110 may optimize synchronization processes, allowing dynamic updates while maintaining structured consistency across various domains.

Additionally, the bidirectional synchronization process facilitated by connectors 114 and the event streams 122 may enable organizations across various domains, such as finance, healthcare, education, and legal services, to manage and analyze distributed data in a seamless fashion. By detecting and standardizing updates in real time, the system 110 may prevent data fragmentation, eliminate redundancy, and maintain up-to-date records across different stakeholders. Additionally, the integration of advanced ML models within parsing, normalization, and terminology mapping stages may further enhance data accuracy and consistency, allowing service providers to operate on a shared, schema-agnostic data representation while preserving interoperability with their proprietary systems.

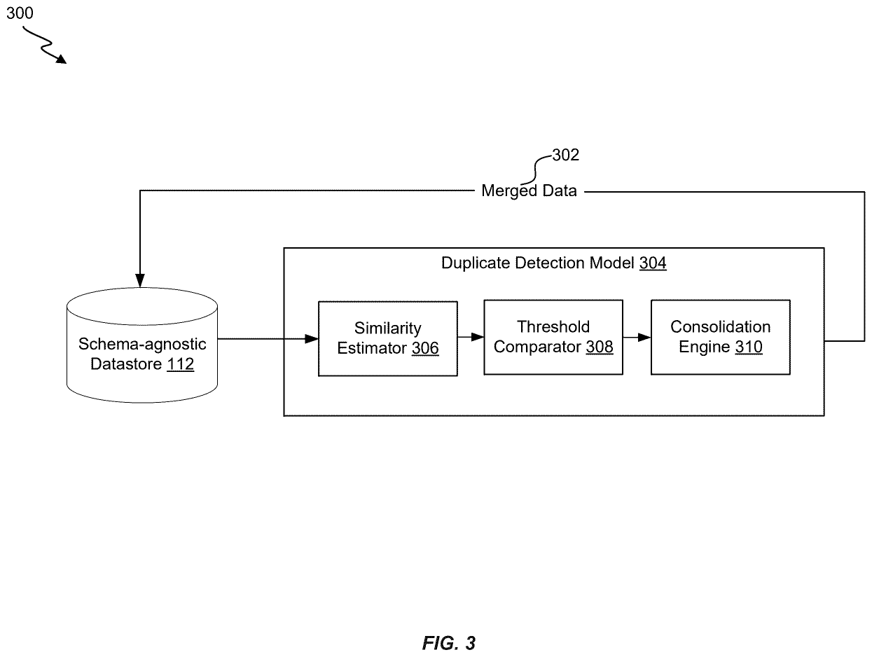

FIG. 3 illustrates an overview diagram of a duplicate detection model 304 to detect whether a user's information is associated one or more times with the same user from the schema-agnostic data 214 according to some aspects of the present disclosure. The duplicate detection model 304 may include a similarity estimator 306, a threshold comparator 308, and a consolidation engine 310. The user information from the schema-agnostic datastore 112 may be accessed by the duplicate detection model 304 to analyze and detect duplicate user information that may be retrieved from multiple datastores 104a-n associated with the same user.

The similarity estimator 306 may process user attributes such as first name, last name, contact details, address, and other personal identifiers to evaluate a degree of similarity between different received records of the same user. To achieve this, ML models such as NLP-based entity matching models, Siamese neural networks, or fuzzy string matching algorithms may be utilized. These models may be trained on historical datasets including labeled instances of duplicate and non-duplicate records, allowing them to learn common variations in a user's information, such as spelling inconsistencies, missing entries, and formatting differences. Additionally, embeddings generated from deep learning models such as Word2Vec, FastText, or BERT may also be used to compare textual data and extract meaningful relationships between different field representations associated with the information of the same user. The degree of the similarity between two or more instances of the same user's information may be represented by a similarity score generated by the similarity estimator 306.

Once a similarity score is generated, the threshold comparator 308 may assess whether the detected similarity meets a predefined confidence threshold to infer that one or more records are duplicate records of the same user. The predefined confidence threshold may represent the minimum required similarity score for the detected duplicates to be classified as duplicates with a high degree of confidence. Various ML techniques, such as logistic regression, support vector machines (SVMs), or ensemble methods like random forests and gradient boosting models, may be used to optimize this classification process. These models may be trained on curated datasets including examples of correctly and incorrectly matched records, enabling them to refine their decision boundaries over time. The threshold comparator 308 may assign a probability score to each detected similarity score and compare it to the predefined confidence threshold. If the similarity score is significantly lower than the threshold, the detected duplicates may be treated as distinct records. However, if the similarity score is close to or exceeds the confidence threshold, the detected data may be considered duplicate entries.

When duplicate records may be identified, the consolidation engine 310 may initiate the process of merging them into a unified instance of a user's information. The consolidation engine 310 may utilize rule-based merging techniques, probabilistic record linkage models, or transformer-based aggregation models to integrate relevant attributes from each duplicate data while minimizing data loss or redundancy. These models may be trained on datasets comprising structured and semi-structured records to learn optimal strategies for consolidating overlapping information. The merged data may retain essential details, including the most complete and up-to-date values for personal attributes, service interactions, and transaction history. In the context of a patient's appointment scheduling scenario, for example, if a patient has multiple appointment records under slightly different name spellings or contact details, the duplicate detection model 304 may recognize the similarity, generate a relatively high similarity score, and trigger the consolidation engine 310 to merge those records. The resulting unified record may accurately reflect the patient's upcoming scheduled appointments, past visit history, and associated medical details across various healthcare providers, removing duplicate records of various patients from the datastores.

Once the duplicate user information is consolidated and merged into a single record or data, it may be stored in the schema-agnostic datastore 112, replacing the fragmented or duplicate entries. This updated data may be propagated to relevant datastores 104a-n via an event-driven bidirectional synchronization (i.e., the connectors 114 and the event streams 122), enabling various service providers 106a-n to access a consistent and de-duplicated version of the user's information. By systematically detecting, evaluating, and consolidating duplicate information elements, the duplicate detection model 304 may optimize data integrity, improve operational efficiency, and enhance the accuracy of user-related transactions and service interactions across the service providers 106a-n.

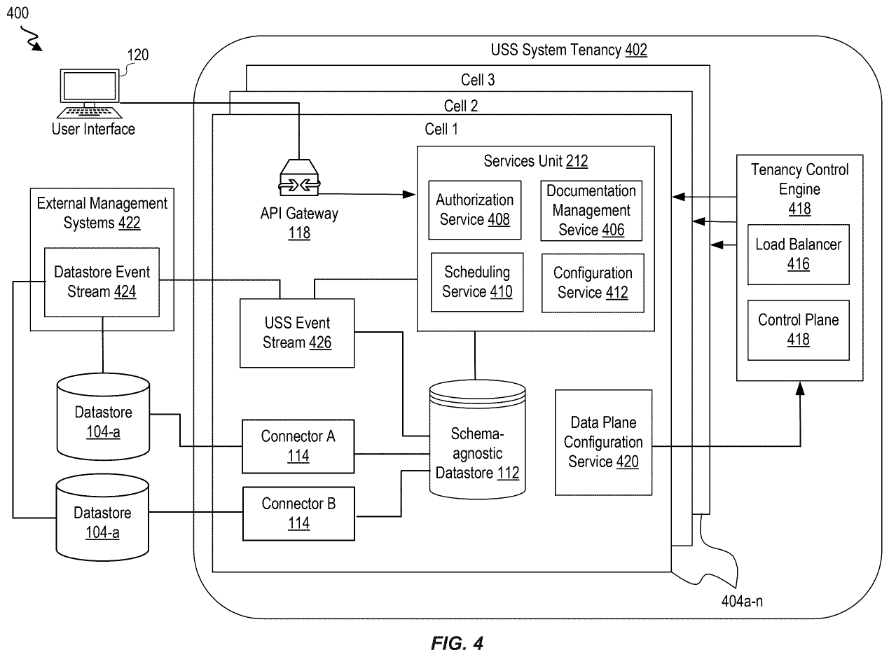

FIG. 4 illustrates a multi-tenancy structure 400 of the unified synchronization system 110 in accordance with some aspects of the present disclosure. The multi-tenancy structure of the unified synchronization system 110 may allow multiple service providers 106a-n to operate within the same infrastructure while maintaining logical separation of their respective data. Instead of deploying an independent instance of the unified synchronization system 110 for each service provider 106, multiple providers may share the same underlying infrastructure, with various mechanisms in place to isolate their data securely.

The multi-tenant architecture as depicted in the system tenancy 402 may include a tenancy control engine 418 that may serve as a centralized layer that manages and coordinates the operation of multiple tenant cells 404a-n. The tenancy control engine 418 may include a load balancer 416, and a control plane 418. Each cell of the multiple tenant cells 404a-n may function as independent infrastructure units that may access all essential components required for processing the data that may be stored in the schema-agnostic datastore 112, such as the API gateway 118, the connectors 202, the event streams 122, and the services unit 116, and a data plane configuration service 420. The services offered by the services unit 116 may include a scheduling service 410, an authorization service 408, a documentation management service 406 and a configuration service 412. Furthermore, the data plane configuration service 420 may be deployed inside each of the tenant cells 404a-n and enables the synchronization of data within the tenant cells with the tenancy control engine 418. The data plane configuration service 420 may periodically interact with the tenancy control engine 418, via an API, to retrieve an updated list of tenants that may need data synchronization. As a part of each tenant cell 404a-n, the data plane configuration service 420 also maintains tenant isolation, enforces secure access policies, and enables seamless data replication across the unified synchronization system 110.

According to some aspects, a tenant cell of the tenant cells 404a-n may host multiple smaller service providers 106a-n, whereas a larger services network with large volumes of data and associated processing requirements may be allocated a dedicated cell. Each service provider 106a-n may be treated as a separate tenant, and requests received by the unified synchronization system 110 may include a tenant identifier appended at the API gateway 118. The tenant identifier may allow the system to associate each request with the correct service provider 106a-n and hence isolate the data. Moreover, each service provider 106a-n may interact with the unified synchronization system 110 using the unique tenant identifier, preventing cross-provider access and maintaining secure data handling practices.

Each record or data within the schema-agnostic datastore 112 may be tagged with the corresponding tenant identifier, preventing unauthorized access across different service providers 104a-n and the processing of incoming requests efficiently while maintaining a tenant's isolation. The unified synchronization system 110 may enforce access control policies through the API gateway 118, which may regulate interactions with the underlying schema-agnostic datastore 112. Furthermore, the system tenancy 402 may incorporate USS event streams 426 (inner event streams of the unified synchronization system) within each cell 404a-n. The USS event streams 426 may facilitate real-time event processing and data synchronization across different components of unified synchronization system 110 as well as the external management systems 422. The external management systems may include the IRCS (integrated revenue cycle suite), hosting various subnet systems such as health information management (HIM), oracle health patient accounting (OHPACT), and common financial clearance (CFC). Additionally, datastore event stream 424 (or EHR event stream) may enable the synchronization of any updates occurring in the datastores 104a-n to be transmitted to the schema-agnostic datastore 112. By interconnecting the services unit 116, the schema-agnostic datastore 112, and the service providers 106a-n, the event streams 122 may enable seamless integration and synchronization of data across the service providers 106a-n.

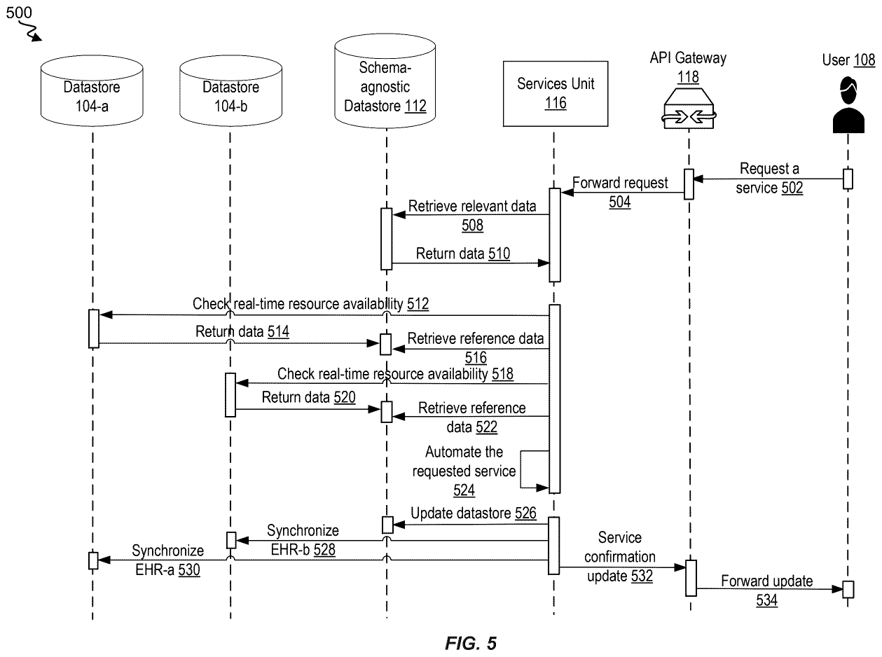

FIG. 5 shows a sequence diagram 500 for processing a service request received from the user 108 via the API gateway 214 according to some aspects of the present disclosure. At process 502, a request may be received from the user 108, that may include a request for a specific action or service, which may pertain to various domains such as healthcare, finance, logistics, legal services, or enterprise resource management. The request may be sent to the API gateway 118, that may act as a centralized entry point and is responsible for handling, routing, and securing requests from the users 108 to the unified synchronization system 110. The API gateway 118 may forward the request to the services unit 116 for further processing, at process 504. The services unit 116 may include various specialized services offered to the user 108 and the service providers 106a-n such as registration service, the scheduling service 410, the authorization service 408, the documentation management service 406, the configuration service 412, billing management service, and/or financial clearance service. Additionally, the services unit 116 may incorporate domain-specific services, such as claims processing in an insurance business, supply chain tracking in logistics business, contract validation in legal services, and compliance auditing in financial transactions, allowing the system to support a wide range of service requests.

The services unit 116 may verify and authorize the user's request by checking the activity data stored in the schema-agnostic datastore 112, at process 508. If the user 108 may already have been registered at the one or more service providers 106a-n, the user's information specific to the user 108 may be sent back to the services unit 116, at process 510. Upon verifying and authenticating the user information, the services unit 116 may identify the one or more service providers 106a-n initially associated with the user 108 (in case the user's information may have been merged or consolidated in the initial duplication detection and standardization processes). At processes 512 and 518, the services unit 116 may check real-time availability of resources from the datastores 104a-n associated with the identified service providers 106a-n. In other words, the system may retrieve the relevant reference data associated with the available providers and the free scheduling timeslots of the user's registered service providers. These resources may correspond to the service-specific assets or personnel necessary for fulfilling the requested service. In a healthcare business, the resources may include medical professionals such as physicians, therapists, and radiologists, as well as diagnostic equipment like MRI machines, X-ray facilities, or operating rooms. In a finance business, the needed resources may involve customer support agents, risk assessment models, or fraud detection algorithms essential for processing financial transactions. Similarly, in a logistics business, resource availability may refer to fleet vehicles, warehouse storage units, or fulfillment center personnel who may be responsible for dispatching goods to customers. In legal services, the system may check for availability of attorneys, case processing time slots, or compliance verification systems that may be necessary for doing contract analysis. Likewise, in an education ecosystem, relevant resources may include available tutors, virtual classroom slots, or digital content licensing required for course access.

At processes 514 and 520, the relevant reference data may be retrieved or returned from the datastores 104a-n, via the set of decoupled connectors 114, and standardized into a schema-agnostic data to be stored in the schema-agnostic datastore 112. Furthermore, at processes 516 and 522, the relevant reference data stored in the schema-agnostic datastore 112 may be retrieved by the services unit 116 for further processing. According to some aspects, the system may present the user 108 (a front-office administrator) with a list of service providers 106a-n and their corresponding available time slots, enabling the user 108 to make an informed decision based on their specific needs and preferences. According to some other aspects, the services unit 116 may analyze the retrieved reference data and automatically execute the requested service with a service provider based on the analyzed reference data, at process 524.

For example, if a financial institution is processing a loan approval request, the services unit 116 may fetch credit history, risk scores, and outstanding liabilities from multiple sources before making an informed evidence-based decision. Similarly, if a legal firm receives a request for validating a contract, the system may retrieve relevant compliance data, applicable legal precedents, and the attorney's availability before proceeding accepting the request for processing. In another example, if a patient wishes to schedule a physiotherapy session with any of the healthcare providers with whom the patient may already have been registered, the services unit 116 may fetch a list of available physiotherapists and their corresponding free appointment slots across the relevant healthcare providers. Based on the retrieved reference data, the system may automatically schedule the appointment with a healthcare provider based on analyzing multiple factors. These factors may include the geographical proximity of the healthcare provider, where the system may prioritize options based on the distance between the user 108 and the provider's location to enhance convenience and accessibility. Additionally, the availability of a requested service within preferred time slots may also be considered, as users with packed and busy schedules, such as working professionals, may prefer appointment slots for noon or evening compared to the morning ones. The system may also consider historical user's preferences, allowing the system to recommend providers based on past interactions, satisfaction ratings, or recurring service selections. Furthermore, the urgency of service provisioning may influence the selection process, where emergency appointments or high-priority requests may be accommodated ahead of routine consultations. Another factor may involve resource optimization and load balancing, allowing the system to intelligently distributed the service requests across multiple available providers to prevent bottlenecks to enhance overall system's efficiency. Several ML models may be used to automate and enhance the automated service execution processes, depending on performing the specific tasks.

At process 526, the services unit 116 may update the user's information (more specifically, the activity data) stored in the schema-agnostic datastore 112 to include details associated with the services that may have been provided. The details may include, for example, the information on the newly scheduled appointment at the healthcare provider, along with the timeslot and name of the practitioner or specialist, and any relevant contextual details. In addition, at processes 528 and 530, the services unit 116 may synchronize the updated user's information across the respective datastores 104a-n that may be associated with the service providers 106a-n, including the service provider where the service has been provided along with the other service providers that may also have been linked to the user 108 (where the user 108 may initially be registered). Moreover, the administration users or front office users of the service providers 106a-n may separately be updated about the performed service via the set of event streams 122.

Finally, At processes 532 and 534, update the user 108 about the confirmation of the service via the API gateway 118. The service providers 106a-, may also be updated via the set of event streams 122.

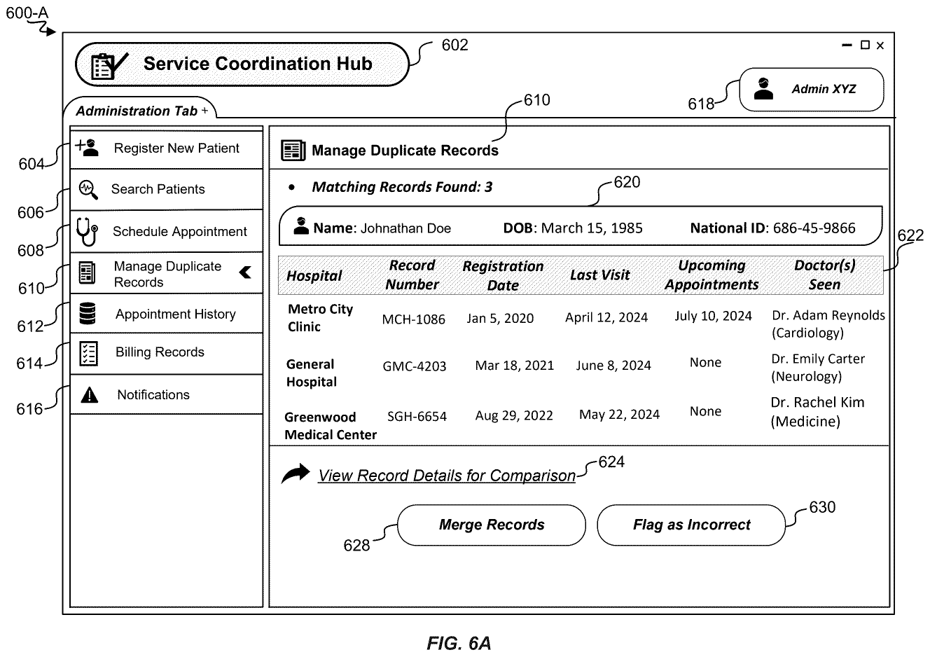

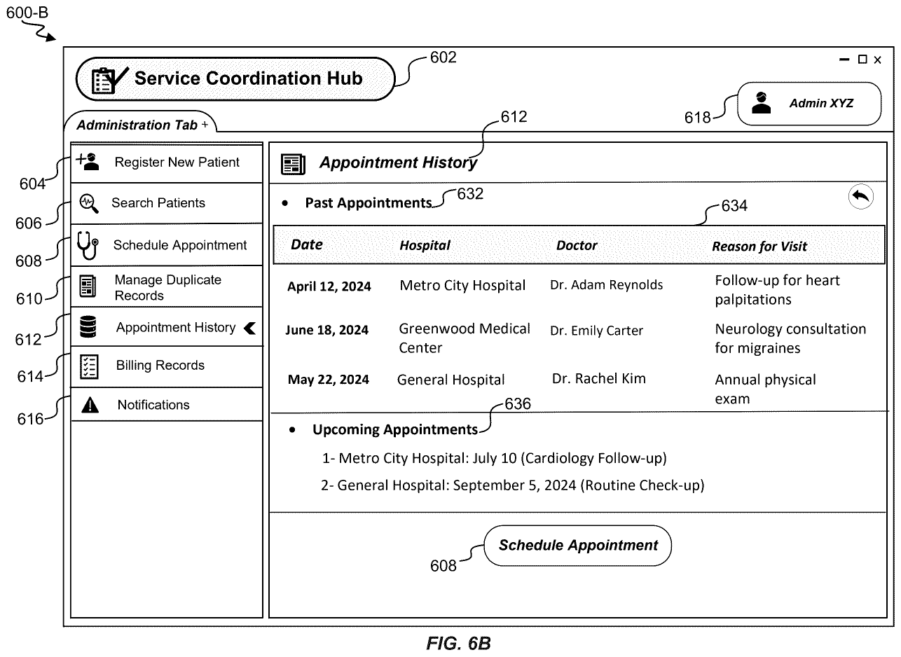

FIG. 6A illustrates an exemplary user interface (UI) 600-A of an authorized user displaying a user dashboard according to some aspects of the present disclosure. The exemplary UI 600-A may display a service coordination hub 602, which may serve as the central interface for managing user-related administration tasks. In the exemplary UI 600-A, the service coordination hub 602 relates to the healthcare domain, depicting an interface or a portal that may be offered by the unified synchronization system 110 to the user 108. The user 108 may be a patient or a front-office administrator of a clinic, hospital, or a healthcare provider. Depending on the entity that may have been logged into the service coordination hub 602, the interface may include an admin-office tab (administration tab) or a patient's tab, allowing the user to access relevant functions of the service coordination hub 602. A profile option 618 may be present within the interface, displaying the name or additional details of the logged-in user, who may be a front-office administrator that may be responsible for handling patient's administration tasks. The profile option 618 may also allow the user 108 to manage user's account settings or switch between different administrative roles.

A sidebar within the administration tab may display various administration options, providing the user 108 with an organized view of available functions. These options may include registering a new patient 604, allowing the user to initiate the registration process for a new patient within the system. Additionally, a search patients 606 option may be available, enabling the user to retrieve an administration data (user information) of registered patients based on specific search parameters such as name, national ID, or medical record number. A schedule appointment 608 option may allow the user to access appointment scheduling functionalities, facilitating the management of patients'appointments across multiple healthcare providers. The manage duplicate records 610 option may provide the user with tools to handle administration data discrepancies, such as identifying and resolving duplicate user records within the system.

Another option, appointment history 612, may allow the user to access comprehensive records of the user 108 in the past or upcoming appointments. The history may include details such as appointment dates, times, the name of the clinic or healthcare facility, and the details of the specialist or practitioner consulted. If the patient is associated with multiple clinics or healthcare providers, the appointment history may consolidate and display all relevant details across different healthcare facilities. This may include information on canceled or rescheduled appointments, the reason for cancellations, consultation notes, prescribed treatments, and follow-up recommendations. Additionally, the system may categorize appointments based on type (e.g., routine check-up, diagnostic procedure, follow-up visit) and provide filters or search functionality to allow users to quickly locate specific records.

The exemplary UI 600-A may dynamically adjust to a display's detailed options when specific administrative functionalities are selected. If the user 108 selects a manage duplicate records 610 option, the UI may present an organized view of duplicate records that may have been detected within the system. The system may analyze the user's information associated with the patients and identify potential duplicate entries based on similarities in registration details. The interface may display the total number of duplicate records associated with a patient, providing the user with an overview of potential data inconsistencies. A details section 620 may present key user information of the patient in question, such as their name (e.g., Johnathan Doe), date of birth (DOB), and national ID. These details may allow the user to quickly verify a patient's identity and assess whether duplicate data may need any further actions.

In addition to the details section, a table 622 may provide an expanded view of the patient's data across multiple datastores 104a-n (or EHRs) associated with different healthcare providers. The table may list the names of healthcare providers where the patient may have been registered, along with the corresponding record numbers assigned to the patient in each respective datastore. The table may also display key historical data, such as the date of initial registration at each healthcare provider, the date of the last visit, and any upcoming appointment dates. Furthermore, the table may include specialist details from the last visit, enabling the user to review the information about a subject's previous interactions with healthcare professionals.

To further assist the user 108 in handling duplicate records, a view of records'details for comparison option 624 may be available within the interface. This option may enable the user 108 to view a side-by-side comparison of the detected duplicate data, particularly focusing on registration details that may have been recorded across multiple datastores 104a-n. By analyzing this comparison, the user 108 may assess whether the duplicate records pertain to the same patient and then according determine the next appropriate action. These actions may include merge records option 628, and flag as incorrect option 630. This process may allow the front-office administrator to maintain accurate and consolidated patient data while preventing discrepancies across various healthcare providers.

FIG. 6B illustrates an exemplary UI 600-B showing the user information of the user 108 associated with multiple service providers 106a-n according to some aspects of the present disclosure. This interface may display the patient's merged data, allowing a consolidated view of past and upcoming appointments. The appointment history 612 may be divided into two sections: a past appointments section 632 and an upcoming appointments section 636, each providing relevant details about the patient's interactions with different clinics, hospitals, or medical institutions.

The past appointments section 632 may list previous medical visits, including the date of each appointment, the name of the hospital or healthcare provider where the visit took place, the attending doctor, and the reason for the consultation. For instance, if a patient had a cardiology follow-up at Metro City Hospital on Apr. 12, 2024, followed by a neurology consultation at Greenwood Medical Center on Jun. 8, 2024, the interface may display this chronological information to offer a better view of the medical history. This organized presentation of past visits may allow the user 108 to quickly assess the patient's medical background and identify patterns in their healthcare utilization.

The upcoming appointments section 636 may present scheduled visits including the date and the name of the hospital where the appointment may take place. For example, if the patient has a cardiology follow-up scheduled at Metro City Hospital on Jul. 10, 2024, and a routine check-up at Starlight General Hospital on Sep. 5, 2024, these details may be displayed within this section. This overview may help the user 108 manage future appointments effectively by identifying potential scheduling conflicts and adjusting plans accordingly.

By analyzing the appointment history 612, the user 108 may determine the most suitable time for booking a new appointment, avoiding overlaps that may lead to logistical challenges or delayed care. Additionally, if an appointment request for the patient is pending, the user 108 may review the patient's existing schedule before approving or modifying the appointment request. This functionality may enable more efficient appointment coordination across multiple service providers 106a-n, enhancing continuity of care and reducing administrative inefficiencies and their associated burden.

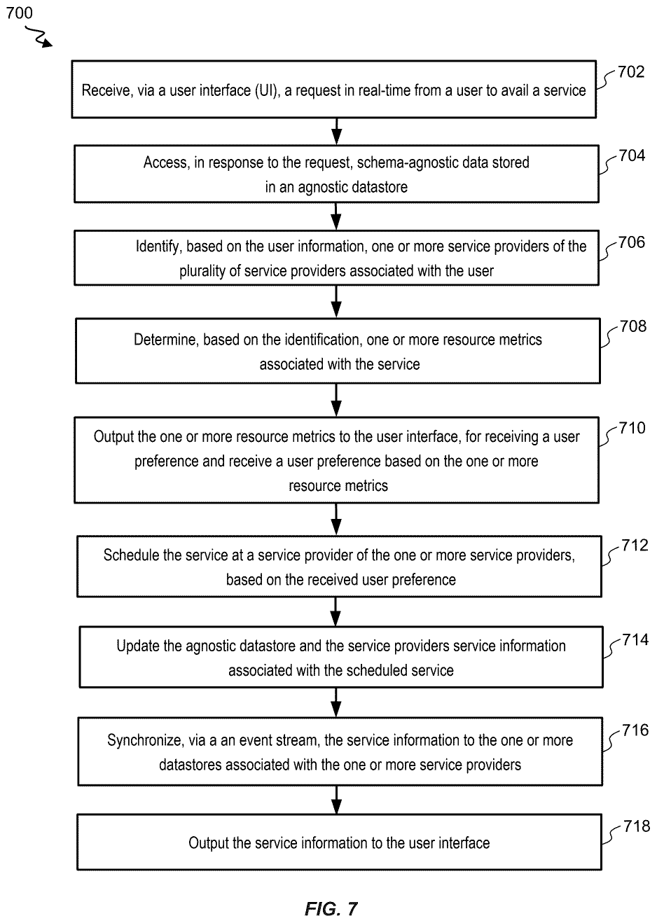

FIG. 7 shows an example flowchart of a system for managing and synchronizing data across multiple datastores according to some aspects of the present disclosure. At block 702, a request may be received from the user 108 to book a service. The service may correspond to a scheduling or a registration service. At block 704, a schema-agnostic data stored in the schema-agnostic datastore 112 may be accessed. Based on the accessed schema-agnostic data, one or more service providers may be identified from a plurality of service providers, at block 706.

At block 708, one or more resource metrics may be determined from the schema-agnostic datastore 112. The resource metric may include a providers'availability, an available time slot, or a resource availability that may be needed while providing the service. The determined one or more resource metrics may be sent to the UI for receiving a user preference, at block 710. The user may select from available service providers having requisite resources for performing the service. Based on the selected user preference, the service may be scheduled at the preferred service provider, at block 712.

At block 714, the schema-agnostic datastore 112, and the selected service provider may be updated using the scheduled service. This update to the service provider may be done by an event stream configured to synchronize updates associated with the request or the service across the service providers. At block 716, the service information associated with the scheduled service may be synchronized to the one or more datastores associated with the one or more service providers via a set of decoupled connectors. Moreover, at block 718, the service information may also be output at the display of the user's interface.

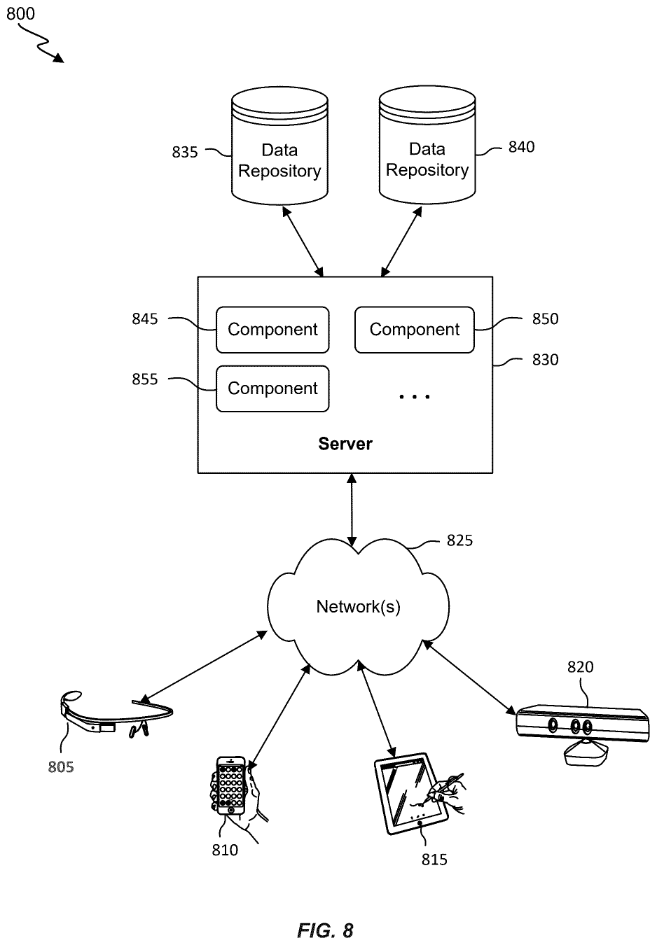

FIG. 8 illustrates a simplified diagram of an example distributed system 800 for a cloud hosting a unified synchronization system 110 system. In the illustrated example, the distributed system 800 includes one or more client computing devices 805, 810, 815, and 820, coupled to a server 830 via one or more communication networks 825. The entity computing devices 805, 810, 815, and 820 may be configured to execute one or more applications interact with the server 830 to access and utilize the unified synchronization platform securely integrated within a cloud environment, such as Oracle cloud integrated with Cohere. Within this framework, the server 830 is configured to host and manage a range of services or software applications, facilitating seamless integration and operation of the unified synchronization system.

In various aspects, the server 830 may extend its capabilities to encompass additional services or software applications. These services may span both virtual and non-virtual environments, enabling a comprehensive and adaptable infrastructure for securely deploying GenAI solutions within the cloud ecosystem. In some respects, these services may be offered as web-based or cloud services, such as under a Software as a Service (SaaS) model to the users of the client computing devices 805, 810, 815, and/or 820. Users operating the client computing devices 805, 810, 815, and/or 820 may in turn utilize one or more client applications to interact with the server 830 to utilize the services provided by these components. Furthermore, client computing devices 805, 810, 815, and/or 820 may in turn utilize one or more client applications to initiate and manage specific tasks or analyses within the unified synchronization system.

In the configuration depicted in FIG. 8, the server 830 may include one or more components 845, 850 and 855 that implement the functions performed by the server 830. These components may include software components that may be executed by one or more processors, hardware components, or combinations thereof. It should be appreciated that various system configurations are possible, which may differ from distributed system 800. The example shown in FIG. 8 is thus one example of a distributed system for implementing an example system and is not intended to be limiting.

Users may initiate requests for the unified synchronization system through client computing devices 805, 810, 815, and/or 820 for inference or other machine-learning tasks. A client device may provide an interface that enables a user of the client device to interact with the unified synchronization system. The client device may also output information to the user via this interface. Although FIG. 8 depicts only four client computing devices, any number of client computing devices may be supported providing scalability and accessibility within the integrated unified synchronization system on the cloud.

The client devices may include various types of computing systems, such as portable handheld devices, general purpose computers, such as personal computers and laptops, workstation computers, wearable devices, gaming systems, thin clients, various messaging devices, sensors or other sensing devices, and the like. These computing devices may run various types and versions of software applications and operating systems (e.g., Microsoft Windows®, Apple Macintosh®, UNIX® or UNIX-like operating systems, Linux or Linux-like operating systems, such as Google Chrome™ OS) including various mobile operating systems (e.g., Microsoft Windows Mobile®, iOS®, Windows Phone®, Android™, BlackBerry®, Palm OS®). Portable handheld devices may include cellular phones, smartphones, (e.g., an iPhone®), tablets (e.g., iPad®), personal digital assistants (PDAs), and the like. Wearable devices may include Google Glass® head mounted display, and other devices. Gaming systems may include various handheld gaming devices, Internet-enabled gaming devices (e.g., a Microsoft Xbox® gaming console with or without a Kinect® gesture input device, Sony PlayStation® system, various gaming systems provided by Nintendo®, and others), and the like. The client devices may be capable of executing various applications, such as various Internet-related apps, communication applications (e.g., E-mail applications, short message service (SMS) applications) and may use various communication protocols.

Network(s) 825 may be any type of network familiar to those skilled in the art that can support data communications using any of a variety of available protocols, including without limitation TCP/IP (transmission control protocol/internet protocol), SNA (systems network architecture), IPX (internet packet exchange), AppleTalk®, and the like. Merely by way of example, network(s) 825 can be a local area network (LAN), networks based on ethernet, token-ring, a wide-area network (WAN), the internet, a virtual network, a virtual private network (VPN), an intranet, an extranet, a public switched telephone network (PSTN), an infra-red network, a wireless network (e.g., a network operating under any of the institute of electrical and electronics (IEEE) 1002.11 suite of protocols, Bluetooth®, and/or any other wireless protocol), and/or any combination of these and/or other networks.

The server 830 may be composed of one or more general purpose computers, specialized server computers (including, by way of example, PC (personal computer) servers, UNIX® servers, mid-range servers, mainframe computers, rack-mounted servers, etc.), server farms, server clusters, or any other appropriate arrangement and/or combination. The server 830 can include one or more virtual machines running virtual operating systems, or other computing architectures involving virtualization, such as one or more flexible pools of logical storage devices that can be virtualized to maintain virtual storage devices for the server. In various aspects, the server 830 may be adapted to run one or more services or software applications that provide the functionality described in the foregoing disclosure.

The computing systems in the server 830 may run one or more operating systems including any of those discussed above, as well as any commercially available server operating system. The server 830 may also run any of a variety of additional server applications and/or mid-tier applications, including HTTP (hypertext transport protocol) servers, FTP (file transfer protocol) servers, CGI (common gateway interface) servers, JAVA® servers, database servers, and the like. Exemplary database servers include without limitation those commercially available from Oracle®, Microsoft®, Sybase®, IBM® (International Business Machines), and the like.

Distributed system 800 may also include one or more data repositories 835, 840. Data repositories 835, 840 may reside in many locations. For example, a data repository used by the server 830 may be local to server 830 or may be remote from the server 830 and in communication with the server 830 via a network-based or dedicated connection. Data repositories 835, 840 may be of different types. In some instances, a data repository used by the server 830 may be a database, for example, a relational database, such as databases provided by Oracle Corporation® and other vendors. One or more of these databases may be adapted to enable storage, update, and retrieval of data to and from the database in response to structured query language (SQL)-formatted commands. In some aspects, one or more data repositories 835, 840 may also be used by applications to store application data. The data repositories used by applications may be of different types, such as, for example, a key-value store repository, an object store repository, or a general storage repository supported by a file system.

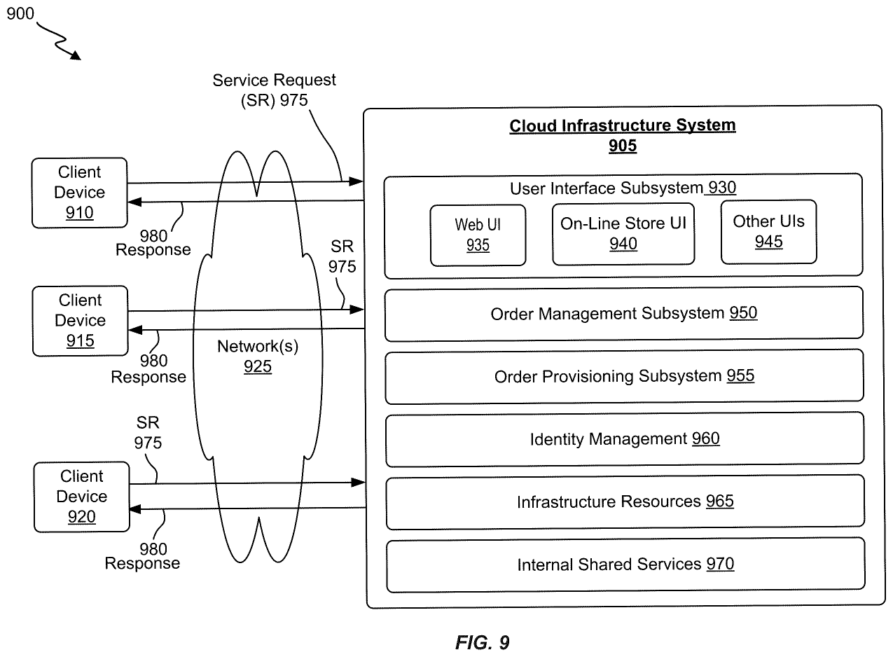

FIG. 9 is a simplified block diagram of a cloud-based system environment in which various services of the server 830 of FIG. 8 may be offered as cloud services, in accordance with certain aspects. In the illustrative example depicted in FIG. 9, cloud infrastructure system 905 may provide one or more cloud services that may be requested by users using one or more client devices 910, 915, and 920. Cloud infrastructure system 905 may comprise one or more computers and/or servers that may include those described for server 930. The computers in cloud infrastructure system 905 may be organized as general-purpose computers, specialized server computers, server farms, server clusters, or any other appropriate arrangement and/or combination.

Network(s) 925 may facilitate communication and exchange of data between client devices 910, 915, and 920 and cloud infrastructure system 905. Network(s) 925 may include one or more networks. The networks may be of the same or different types. Network(s) 925 may support one or more communication protocols, including wired and/or wireless protocols, for facilitating the communications.

The illustrative example depicted in FIG. 9 is only one example of a cloud infrastructure system 905 and is not intended to be limiting. It should be appreciated that, in some other aspects, cloud infrastructure system 905 may have more or fewer components than those depicted in FIG. 9, may combine two or more components, or may have a different configuration or arrangement of components. For example, although FIG. 9 depicts three client computing devices, any number of client computing devices may be supported in alternative aspects.

The term cloud service is generally used to refer to a service that is made available to users on demand and via a communication network, such as the internet by systems (e.g., cloud infrastructure system 905) of a service provider. Typically, in a public cloud environment, servers and systems that make up the cloud service provider's system are different from the client's own on-premises servers and systems. The cloud service provider's systems are managed by the cloud service provider. Clients can thus avail themselves of cloud services provided by a cloud service provider without having to purchase separate licenses, support, or hardware and software resources for the services. For example, a cloud service provider's system may host an application, and a user may, via a network 925 (e.g., the internet), on demand, order and use the application without the user having to buy infrastructure resources for executing the application. Cloud services are designed to provide easy, scalable access to applications, resources, and services. Several providers offer cloud services. For example, several cloud services are offered by Oracle Corporation® of Redwood Shores, California, such as middleware services, database services, Java cloud services, and others.

In certain aspects, cloud infrastructure system 905 may provide one or more cloud services using different models, such as under a Software as a Service (SaaS) model, a Platform as a Service (PaaS) model, an Infrastructure as a Service (IaaS) model, and others, including hybrid service models. Cloud infrastructure system 905 may include a suite of applications, middleware, databases, and other resources that enable provision of the various cloud services.

A SaaS model enables an application or software to be delivered to a client over a communication network like the Internet, as a service, without the client having to buy the hardware or software for the underlying application. For example, a SaaS model may be used to provide clients access to on-demand applications that are hosted by cloud infrastructure system 905. Examples of SaaS services provided by Oracle Corporation® include, without limitation, various services for human resources/capital management, client relationship management (CRM), enterprise resource planning (ERP), supply chain management (SCM), enterprise performance management (EPM), analytics services, social applications, and others.

An IaaS model is generally used to provide infrastructure resources (e.g., servers, storage, hardware, and networking resources) to a client as a cloud service to provide elastic compute and storage capabilities. Various IaaS services are provided by Oracle Corporation®.

A PaaS model is generally used to provide, as a service, platform and environment resources that enable clients to develop, run, and manage applications and services without the client having to procure, build, or maintain such resources. Examples of PaaS services provided by Oracle Corporation® include, without limitation, Oracle Java Cloud Service (JCS), Oracle Database Cloud Service (DBCS), data management cloud service, various application development solutions services, and others.

Cloud services are generally provided on an on-demand self-service basis, subscription-based, elastically scalable, reliable, universally available, and secure manner. For example, a client, via a subscription order, may order one or more services provided by cloud infrastructure system 905. Cloud infrastructure system 905 then performs processing to provide the services requested in the client's subscription order. Cloud infrastructure system 905 may be configured to provide one or even multiple cloud services.

Cloud infrastructure system 905 may provide cloud services via different deployment models. In a public cloud model, cloud infrastructure system 905 may be owned by a third-party cloud services provider and the cloud services are offered to any general public client, where the client can be an individual or an enterprise. In certain other aspects, under a private cloud model, cloud infrastructure system 905 may be operated within an organization (e.g., within an enterprise organization) and services provided to clients that are within the organization. For example, the clients may be various departments of an enterprise, such as the Human Resources department, the payroll department, etc. or even individuals within the enterprise. In certain other aspects, under a community cloud model, the cloud infrastructure system 905 and the services provided may be shared by several organizations in a related community. Various other models, such as hybrids of the above-mentioned models may also be used.

Client devices 910, 915, and 920 may be of several types (such as client computing devices 905, 910, 915, and 920 depicted in FIG. 9) and may be capable of operating one or more client applications. A user may use a client device to interact with cloud infrastructure system 905, such as to request a service provided by cloud infrastructure system 905. For instance, a user might employ a client device to execute real-time data querying operations within the cloud. A client may use a client device, such as a laptop to interact with the unified synchronization system integrated within cloud infrastructure system. The client may request GPU-accelerated computing instances of the cloud for training deep learning models. The cloud may provide the necessary resources, and the subject client may monitor and manage the training process through the laptop. Upon completion, the client may retrieve the trained models and results.

In certain aspects, to facilitate efficient provisioning of these resources for supporting the various cloud services provided by cloud infrastructure system 905 for different clients, the resources may be bundled into sets of resources or resource modules. Each resource module or pod may comprise a pre-integrated and optimized combination of resources of one or more types. In certain aspects, different pods may be pre-provisioned for different types of cloud services. For example, a first set of pods may be provisioned for a database service, a second set of pods, which may include a different combination of resources than a pod in the first set of pods, may be provisioned for Java service, and the like. For some services, the resources allocated for provisioning the services may be shared between the services.

Cloud infrastructure system 905 may comprise multiple subsystems. These subsystems may be implemented in software, or hardware, or combinations thereof. As depicted in FIG. 9, the subsystems may include a user interface subsystem 930 that enables users or clients of cloud infrastructure system 905 to interact with cloud infrastructure system 905. User interface subsystem 930 may include various interfaces, such as a web user interface 935, an online store interface 940 where cloud services provided by cloud infrastructure system 905 are advertised and are purchasable by a consumer, and other interfaces 945. For example, a client may, using a client device, request (service request 975) one or more services provided by cloud infrastructure system 905 using one or more of interfaces 935, 940, and 945. For example, a client may access the online store, browse cloud services offered by cloud infrastructure system 905, and place a subscription order for one or more services offered by cloud infrastructure system 905 that the client wishes to subscribe to. The service request may include information identifying the client and one or more services that the client desires to subscribe to. For example, a client may place a subscription order for a Chabot related service offered by cloud infrastructure system 905. As part of the order, the client may provide information identifying for input (e.g., utterances).

In certain aspects, such as the illustrative example depicted in FIG. 9, cloud infrastructure system 905 may comprise an order management subsystem (OMS) 950 that is configured to process the new order. As part of this processing, OMS 950 may be configured to: create an account for the client, if not done already; receive billing and/or accounting information from the client that is to be used for billing the client for providing the requested service to the client; verify the client information; upon verification, book the order for the client; and orchestrate various workflows to prepare the order for provisioning.

Once properly validated, OMS 950 may then invoke the order provisioning subsystem (OPS) 955 that is configured to provision resources for the order including processing, memory, and networking resources. The provisioning may include allocating resources for the order and configuring the resources to facilitate the service requested by the client order. The way resources are provisioned for an order and the type of the provisioned resources may depend upon the type of cloud service that has been ordered by the client. For example, according to one workflow, OPS 955 may be configured to determine the particular cloud service being requested and identify a number of pods that may have been pre-configured for that particular cloud service. The number of pods that are allocated for an order may depend upon the size/amount/level/scope of the requested service. For example, the number of pods to be allocated may be determined based upon the number of users to be supported by the service, the duration of time for which the service is being requested, and the like. The allocated pods may then be customized for the requesting client for providing the requested service.

Cloud infrastructure system 905 may itself internally use services 970 that are shared by different components of cloud infrastructure system 905 and which facilitate the provisioning of services by cloud infrastructure system 905. These internal shared services may include, without limitation, a security and identity service, an integration service, an enterprise repository service, an enterprise manager service, a virus scanning and whitelist service, a high availability, backup and recovery service, service for enabling cloud support, an email service, a notification service, a file transfer service, and the like. As depicted in the illustrative example in FIG. 9, cloud infrastructure system 905 may include infrastructure resources 965 that can be utilized for facilitating the provision of various cloud services offered by cloud infrastructure system 905. Infrastructure resources 965 may include, for example, processing resources, storage or memory resources, networking resources, and the like. Cloud infrastructure system 905 may send a response or notification 910 to the requesting client to indicate when the requested service is now ready for use. In some instances, information (e.g., a link) may be sent to the client that enables the client to start using and availing the benefits of the requested services.

Cloud infrastructure system 905 may provide services to multiple clients in parallel. Cloud infrastructure system 905 may store information for these clients, including possibly proprietary information. In certain aspects, cloud infrastructure system 905 comprises an identity management subsystem (IMS) 960 that is configured to manage client's information and provide the separation of the managed information such that information related to one client is not accessible by another client. IMS 960 may be configured to provide various security-related services, such as identity services, such as information access management, authentication and authorization services, services for managing client identities and roles and related capabilities, and the like.

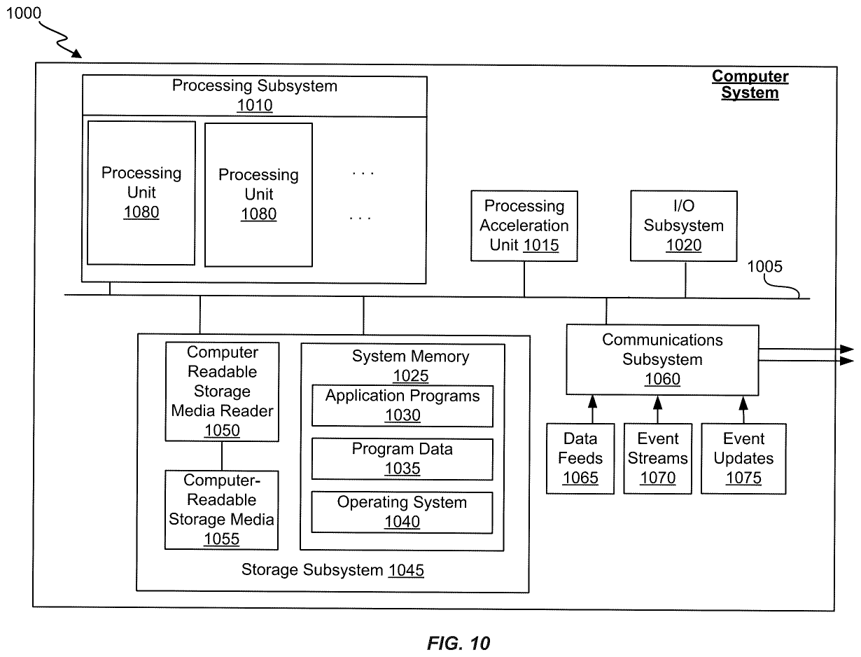

FIG. 10 illustrates an exemplary computer system 1000 that may be used to implement certain aspects of the present disclosure. For example, a computer system 1000 may facilitate the integration of a Subject management system with the cloud by provisioning and configuring resources, managing data, implementing security measures, monitoring performance, and enabling scalability. It may serve as the foundational infrastructure, enabling seamless deployment and operation of AI applications within the cloud environment while providing flexibility and scalability to adapt to changing computational demands efficiently. In some aspects, computer system 1000 may be used to implement various servers as described above. As shown in FIG. 10, computer system 1000 may include various subsystems including a processing subsystem 1010 that communicates with a few other subsystems via a bus subsystem 1005. These other subsystems may include a processing acceleration unit 1015, and I/O subsystem 1020, a storage subsystem 1045, and a communications subsystem 1060. Storage subsystem 1045 may include non-transitory computer-readable storage media including storage media 1055 and a system memory 1025.

Bus subsystem 1005 provides a mechanism for letting the various components and subsystems of computer system 1000 communicate with each other as intended. Although bus subsystem 1005 is shown schematically as a single bus, alternative aspects of the bus subsystem may utilize multiple buses. Bus subsystem 1005 may be any of several types of bus structures including a memory bus or memory controller, a peripheral bus, a local bus using any of a variety of bus architectures, and the like. For example, such architectures may include an industry standard architecture (ISA) bus, micro channel architecture (MCA) bus, enhanced ISA (EISA) bus, video electronics standards association (VESA) local bus, and peripheral component interconnect (PCI) bus, which can be implemented as a Mezzanine bus manufactured to the IEEE P1386.1 standard, and the like.

Processing subsystem 1010 controls the operation of computer system 1000 and may comprise one or more processors, application specific integrated circuits (ASICs), or field programmable gate arrays (FPGAs). The processors may include single core or multicore processors. The processing resources of computer system 1000 can be organized into one or more processing units 1080, 1080, etc. A processing unit may include one or more processors, one or more cores from the same or different processors, a combination of cores and processors, or other combinations of cores and processors. In some aspects, processing subsystem 1010 can include one or more special purpose co-processors, such as graphics processors, digital signal processors (DSPs), or the like. In some aspects, some or all of the processing units of processing subsystem 1010 can be implemented using customized circuits, such as application specific integrated circuits (ASICs), or field programmable gate arrays (FPGAs).

In some aspects, the processing units in processing subsystem 1010 can execute instructions stored in system memory 1025 or on computer readable storage media 1055. In various aspects, the processing units can execute a variety of programs or code instructions and can maintain multiple concurrently executing programs or processes. At any given time, some, or all of the program code to be executed can be resident in system memory 1025 and/or on computer-readable storage media 1055 including potentially on one or more storage devices. Through suitable programming, processing subsystem 1010 can provide various functionalities described above. In instances where computer system 1000 is executing one or more virtual machines, one or more processing units may be allocated to each virtual machine.

In certain aspects, a processing acceleration unit 1015 may optionally be provided for performing customized processing or for off-loading some of the processing performed by processing subsystem 1010 to accelerate the overall processing performed by computer system 1000.

I/O subsystem 1020 may include devices and mechanisms for inputting information to computer system 1000 and/or for outputting information from or via computer system 1000. In general, use of the term input device is intended to include all possible types of devices and mechanisms for inputting information to computer system 1000. User interface input devices may include, for example, a keyboard, pointing devices, such as a mouse or trackball, a touchpad or touch screen incorporated into a display, a scroll wheel, a click wheel, a dial, a button, a switch, a keypad, audio input devices with voice command recognition systems, microphones, and other types of input devices. User interface input devices may also include motion sensing and/or gesture recognition devices, such as the Microsoft Kinect® motion sensor that enables users to control and interact with an input device, the Microsoft Xbox® 360 game controller, devices that provide an interface for receiving input using gestures and spoken commands. User interface input devices may also include eye gesture recognition devices, such as the Google Glass® blink detector that detects eye activity (e.g., “blinking” while taking pictures and/or making a menu selection) from users and transforms the eye gestures as inputs to an input device (e.g., Google Glass®). Additionally, user interface input devices may include voice recognition sensing devices that enable users to interact with voice recognition systems (e.g., Siri® navigator) through voice commands.

Other examples of user interface input devices include, without limitation, three dimensional (3D) mice, joysticks or pointing sticks, gamepads and graphic tablets, and audio/visual devices, such as speakers, digital cameras, digital camcorders, portable media players, webcams, image scanners, fingerprint scanners, barcode reader 3D scanners, 3D printers, laser rangefinders, and eye gaze tracking devices. Additionally, user interface input devices may include, for example, medical imaging input devices, such as computed tomography, magnetic resonance imaging, position emission tomography, and medical ultrasonography devices. User interface input devices may also include, for example, audio input devices, such as MIDI keyboards, digital musical instruments, and the like.

In general, use of the term output device is intended to include all possible types of devices and mechanisms for outputting information from computer system 1000 to a user or other computer. User interface output devices may include a display subsystem, indicator lights, or non-visual displays, such as audio output devices, etc. The display subsystem may be a cathode ray tube (CRT), a flat-panel device, such as that using a liquid crystal display (LCD) or plasma display, a projection device, a touch screen, and the like. For example, user interface output devices may include, without limitation, a variety of display devices that visually convey text, graphics, and audio/video information, such as monitors, printers, speakers, headphones, automotive navigation systems, plotters, voice output devices, and modems.

Storage subsystem 1045 provides a repository or data store for storing information and data that is used by computer system 1000. Storage subsystem 1045 provides a tangible non-transitory computer-readable storage medium for storing the basic programming and data constructs that provide the functionality of some aspects. Storage subsystem 1045 may store software (e.g., programs, code modules, instructions) that when executed by processing subsystem 1010 provides the functionality described above. The software may be executed by one or more processing units of processing subsystem 1010. Storage subsystem 1045 may also provide a repository for storing data used in accordance with the teachings of this disclosure.

Storage subsystem 1045 may include one or more non-transitory memory devices, including volatile and non-volatile memory devices. As shown in FIG. 10, storage subsystem 1045 includes a system memory 1025 and a computer-readable storage media 1055. System memory 1025 may include a number of memories including a volatile main random-access memory (RAM) for storage of instructions and data during program execution and a non-volatile read only memory (ROM) or flash memory in which fixed instructions are stored. In some implementations, a basic input/output system (BIOS), containing the basic routines that help to transfer information between elements within computer system 1000, such as during start-up, may typically be stored in the ROM. The RAM typically contains data and/or program modules that are presently being operated and executed by processing subsystem 1010. In some implementations, system memory 1025 may include multiple different types of memory, such as static random-access memory (SRAM), dynamic random-access memory (DRAM), and the like.

By way of example, and not limitation, as depicted in FIG. 10, system memory 1025 may load application programs 1030 that are being executed, which may include various applications, such as Web browsers, mid-tier applications, relational database management systems (RDBMS), etc., program data 1035, and an operating system 1040. By way of example, operating system 1040 may include various versions of Microsoft Windows®, Apple Macintosh®, and/or Linux operating systems, a variety of commercially-available UNIX® or UNIX-like operating systems (including without limitation the variety of GNU/Linux operating systems, the Google Chrome® OS, and the like) and/or mobile operating systems, such as iOS, Windows® Phone, Android® OS, BlackBerry® OS, Palm® OS operating systems, and others.

Computer-readable storage media 1055 may store programming and data constructs that provide the functionality of some aspects. Computer-readable media 1055 may provide storage of computer-readable instructions, data structures, program modules, and other data for computer system 1000. Software (programs, code modules, instructions) that, when executed by processing subsystem 1010 provides the functionality described above, may be stored in storage subsystem 1045. By way of example, computer-readable storage media 1055 may include non-volatile memory, such as a hard disk drive, a magnetic disk drive, an optical disk drive, such as a CD ROM, digital video disc (DVD), a Blu-Ray® disk, or other optical media. Computer-readable storage media 1055 may include, but is not limited to, Zip® drives, flash memory cards, universal serial bus (USB) flash drives, secure digital (SD) cards, DVD disks, digital video tape, and the like. Computer-readable storage media 1055 may also include, solid-state drives (SSD) based on non-volatile memory, such as flash-memory based SSDs, enterprise flash drives, solid state ROM, and the like, SSDs based on volatile memory, such as solid-state RAM, dynamic RAM, static RAM, dynamic random access memory (DRAM)-based SSDs, magneto resistive RAM (MRAM) SSDs, and hybrid SSDs that use a combination of DRAM and flash memory based SSDs.

In certain aspects, storage subsystem 1045 may also include a computer-readable storage media reader 1050 that can further be connected to computer-readable storage media 1055. The computer-readable storage media reader 1050 may receive and be configured to read data from a memory device, such as a disk, a flash drive, etc.

In certain aspects, computer system 1000 may support virtualization technologies, including but not limited to virtualization of processing and memory resources. For example, computer system 1000 may provide support for executing one or more virtual machines. In certain aspects, computer system 1000 may execute a program, such as a hypervisor that facilitated the configuring and managing of the virtual machines. Each virtual machine may be allocated memory, compute (e.g., processors, cores), I/O, and networking resources. Each virtual machine generally runs independently of the other virtual machines. A virtual machine typically runs its own operating system, which may be the same as or different from the operating systems executed by other virtual machines executed by computer system 1000. Accordingly, multiple operating systems may potentially be run concurrently by computer system 1000.

Communications subsystem 1060 provides an interface to other computer systems and networks. Communications subsystem 1060 serves as an interface for receiving data from and transmitting data to other systems from computer system 1000. For example, communications subsystem 1060 may enable computer system 1000 to establish a communication channel to one or more client devices via the Internet for receiving and sending information from and to the client devices. For example, the communication subsystem may be used to transmit a response to a user regarding the inquiry for a Chatbot.

Communication subsystem 1060 may support both wired and/or wireless communication protocols. For example, in certain aspects, communications subsystem 1060 may include radio frequency (RF) transceiver components for accessing wireless voice and/or data networks (e.g., using cellular telephone technology, advanced data network technology, such as 3G, 4G or EDGE (enhanced data rates for global evolution), Wi-Fi (IEEE 802.XX family standards, or other mobile communication technologies, or any combination thereof), global positioning system (GPS) receiver components, and/or other components. In some aspects, communications subsystem 1060 can provide wired network connectivity (e.g., Ethernet) in addition to or instead of a wireless interface.

Communication subsystem 1060 can receive and transmit data in various forms. For example, in some aspects, in addition to other forms, communications subsystem 1060 may receive input communications in the form of data feeds 1065 such as structured and/or unstructured data feeds, event streams 1070, event updates 1075, and the like. For example, communications subsystem 1060 may be configured to receive (or send) data feeds 1065 in real-time from users of social media networks and/or other communication services, such as Twitter® feeds, Facebook® updates, web feeds, such as Rich Site Summary (RSS) feeds, and/or real-time updates from one or more third party information sources.

In certain aspects, communications subsystem 1060 may be configured to receive data in the form of continuous data streams, which may include event streams 1070 of real-time events and/or event updates 1075, that may be continuous or unbounded in nature with no explicit end. Examples of applications that generate continuous data may include, for example, sensor data applications, financial tickers, network performance measuring tools (e.g., network monitoring and traffic management applications), clickstream analysis tools, automobile traffic monitoring, and the like.

Communications subsystem 1060 may also be configured to communicate data from computer system 1000 to other computer systems or networks. The data may be communicated in various forms, such as structured and/or unstructured data feeds 1065, event streams 1070, event updates 1075, and the like to one or more databases that may be in communication with one or more streaming datastore computers coupled to computer system 1000.

Computer system 1000 can be one of various types, including a handheld portable device (e.g., an iPhone® cellular phone, an iPad® computing tablet, a personal digital assistant (PDA)), a wearable device (e.g., a Google Glass® head mounted display), a personal computer, a workstation, a mainframe, a kiosk, a server rack, or any other data processing system. Due to the ever-changing nature of computers and networks, the description of computer system 1000 depicted in FIG. 10 is intended only as a specific example. Many other configurations having more or fewer components than the system depicted in FIG. 10 are possible. Based on the disclosure and teachings provided herein, a person of ordinary skill in art can appreciate other ways and/or methods to implement the various aspects.

Some embodiments of the present disclosure include a system including one or more data processors. In some embodiments, the system includes a non-transitory computer readable storage medium containing instructions which, when executed on the one or more data processors, cause the one or more data processors to perform part or all of one or more methods and/or part or all of one or more processes disclosed herein. Some embodiments of the present disclosure include a computer-program product tangibly embodied in a non-transitory machine-readable storage medium, including instructions configured to cause one or more data processors to perform part or all of one or more methods and/or part or all of one or more processes disclosed herein.

The terms and expressions which have been employed are used as terms of description and not of limitation, and there is no intention in the use of such terms and expressions of excluding any equivalents of the features shown and described or portions thereof, but it is recognized that various modifications are possible within the scope of the invention claimed. Thus, it should be understood that although the present invention as claimed has been specifically disclosed by embodiments and optional features, modification, and variation of the concepts herein disclosed may be resorted to by those skilled in the art, and that such modifications and variations are considered to be within the scope of this invention as defined by the appended claims.

The present description provides preferred exemplary embodiments only, and is not intended to limit the scope, applicability or configuration of the disclosure. Rather, the description of the preferred exemplary embodiments will provide those skilled in the art with an enabling description for implementing various embodiments. It is understood that various changes may be made in the function and arrangement of elements without departing from the spirit and scope as set forth in the appended claims.

Specific details are given in the following description to provide a thorough understanding of the embodiments. However, it will be understood that the embodiments may be practiced without these specific details. For example, circuits, systems, networks, processes, and other components may be shown as components in block diagram form in order not to obscure the embodiments in unnecessary detail. In other instances, well-known circuits, processes, algorithms, structures, and techniques may be shown without unnecessary detail in order to avoid obscuring the embodiments.

To be claimed:

1. A computer-implemented method comprising:
receiving, via a user interface (UI), a request from a user to avail a service, wherein the request corresponds to a scheduling request;
accessing, in response to the request, schema-agnostic data stored in a schema-agnostic datastore, wherein the schema-agnostic data is generated via a set of decoupled connectors by:
retrieving information from a plurality of datastores associated with a plurality of service providers, wherein the information includes service provider information and user information;
generating, based on the retrieved information, normalized data by using one or more encoding models;
generating transformed data by applying an ontology-based terminology mapping to the normalized data associated with each datastore of the plurality of datastores;
detecting duplicate user information associated with the user from the transformed data by using a record linkage model; and
generating merged data from the detected duplicate user information;
identifying, based on the schema-agnostic data, one or more service providers of the plurality of service providers associated with the user;
determining, from the identified one or more service providers, a plurality of resource metrics associated with the service, wherein the plurality of resource metrics include a provider availability, a time slot availability, or a resource availability;
outputting the plurality of resource metrics to the user interface;
receiving a user preference associated with the plurality of resource metrics;
scheduling the service at a service provider of the one or more service providers based on the received user preference;
updating the schema-agnostic datastore with a service information associated with the scheduled service, wherein the service information includes service provider details, contextual information or a scheduled time associated with the scheduled service;
synchronizing the scheduled service to the one or more service providers via a set of event streams, wherein the set of event streams detect and propagate real-time updates associated with the request; and
outputting the service information to the user interface.

2. The computer-implemented method of claim 1, wherein the one or more encoding models include a word embeddings (Word2Vec, GloVe) model, a one-hot encoding model, or a label encoding model.

3. The computer-implemented method of claim 1, wherein the ontology-based terminology mapping includes:
identifying a set of terminologies from the normalized data using transformer-based language models including BERT, BioBERT, or SciBERT; and
generating transformed data by mapping the identified set of terminologies to a predefined, standardized terminology using one or more machine-learning models including BERTMap, OntoAlign, DeepMap, TransE, ComplEx, or RotatE.

4. The computer-implemented method of claim 1, wherein the record linkage model includes a support vector machine (SVM), a pairwise logistic regression model, expectation-maximization (EM) algorithm, or a Siamese network model.

5. The computer-implemented method of claim 1, wherein an event stream of the set of event streams is a transactional event queue (TEQ) that comprises:
an event listener that detects real-time updates associated with the request; and
a message broker that delivers the detected real-time updates to the plurality of service providers.

6. The computer-implemented method of claim 1, further comprising:
detecting, via an event stream of the set of event streams, an update within a datastore of the plurality of datastores, wherein the update is associated with at least one of: registering the user or scheduling the service; and
synchronizing, via the set of decoupled connectors, the update to the schema-agnostic datastore.

7. The computer-implemented method of claim 1, further comprising:
receiving a request via a user interface, wherein the request comprises a registration request of registering the user at one or more service providers of the plurality of service providers;
registering the user based on the registration request by adding registration data associated with the user to the schema-agnostic datastore; and
synchronizing, via the set of event streams, the added registration data associated with the user to one or more datastores associated with the one or more service providers.

8. A system comprising:
one or more processors; and
one or more non-transitory computer-readable media storing instructions, which, when executed by the system, cause the system to perform a set of operations including:
receiving, via a user interface (UI), a request from a user to avail a service, wherein the request corresponds to a scheduling request;
accessing, in response to the request, schema-agnostic data stored in a schema-agnostic datastore, wherein the schema-agnostic data is generated via a set of decoupled connectors by:
retrieving information from a plurality of datastores associated with a plurality of service providers, wherein the information includes service provider information and user information;
generating, based on the retrieved information, normalized data by using one or more encoding models;
generating transformed data by applying an ontology-based terminology mapping to the normalized data associated with each datastore of the plurality of datastores;
detecting duplicate user information associated with the user from the transformed data by using a record linkage model; and
generating merged data from the detected duplicate user information;
identifying, based on the schema-agnostic data, one or more service providers of the plurality of service providers associated with the user;
determining, from the identified one or more service providers, a plurality of resource metrics associated with the service, wherein the plurality of resource metrics include a provider availability, a time slot availability, or a resource availability;
outputting the plurality of resource metrics to the user interface;
receiving a user preference associated with the plurality of resource metrics;
scheduling the service at a service provider of the one or more service providers based on the received user preference;
updating the schema-agnostic datastore with a service information associated with the scheduled service, wherein the service information includes service provider details, contextual information or a scheduled time associated with the scheduled service;
synchronizing the scheduled service to the one or more service providers via a set of event streams, wherein the set of event streams detect and propagate real-time updates associated with the request; and
outputting the service information to the user interface.

9. The system of claim 8, wherein the one or more encoding models include a word embeddings (Word2Vec, GloVe) model, a one-hot encoding model, or a label encoding model.

10. The system of claim 8, wherein the ontology-based terminology mapping includes:
identifying a set of terminologies from the normalized data using transformer-based language models including BERT, BioBERT, or SciBERT; and
generating transformed data by mapping the identified set of terminologies to a predefined, standardized terminology using one or more machine-learning models including BERTMap, OntoAlign, DeepMap, TransE, ComplEx, or RotatE.

11. The system of claim 8, wherein the record linkage model includes a support vector machine (SVM), a pairwise logistic regression model, expectation-maximization (EM) algorithm, or a Siamese network model.

12. The system of claim 8, wherein an event stream of the set of event streams is a transactional event queue (TEQ) that comprises:
an event listener that detects real-time updates associated with the request; and
a message broker that delivers the detected real-time updates to the plurality of service providers.

13. The system of claim 8, wherein the set of operations further includes:
detecting, via an event stream of the set of event streams, an update within a datastore of the plurality of datastores, wherein the update is associated with at least one of: registering the user or scheduling the service; and
synchronizing, via the set of decoupled connectors, the update to the schema-agnostic datastore.

14. The system of claim 8, wherein the set of operations further includes:
receiving a request via a user interface, wherein the request comprises a registration request of registering the user at one or more service providers of the plurality of service providers;
registering the user based on the registration request by adding registration data associated with the user to the schema-agnostic datastore; and
synchronizing, via the set of event streams, the added registration data associated with the user to one or more datastores associated with the one or more service providers.

15. A computer-program product tangibly embodied in a non-transitory machine-readable storage medium, including instructions configured to cause one or more data processors to perform part a set of operations including:
receiving, via a user interface (UI), a request from a user to avail a service, wherein the request corresponds to a scheduling request;
accessing, in response to the request, schema-agnostic data stored in a schema-agnostic datastore, wherein the schema-agnostic data is generated via a set of decoupled connectors by:
retrieving information from a plurality of datastores associated with a plurality of service providers, wherein the information includes service provider information and user information;
generating, based on the retrieved information, normalized data by using one or more encoding models;
generating transformed data by applying an ontology-based terminology mapping to the normalized data associated with each datastore of the plurality of datastores;
detecting duplicate user information associated with the user from the transformed data by using a record linkage model; and
generating merged data from the detected duplicate user information;
identifying, based on the schema-agnostic data, one or more service providers of the plurality of service providers associated with the user;
determining, from the identified one or more service providers, a plurality of resource metrics associated with the service, wherein the plurality of resource metrics include a provider availability, a time slot availability, or a resource availability;
outputting the plurality of resource metrics to the user interface;
receiving a user preference associated with the plurality of resource metrics;
scheduling the service at a service provider of the one or more service providers based on the received user preference;
updating the schema-agnostic datastore with a service information associated with the scheduled service, wherein the service information includes service provider details, contextual information or a scheduled time associated with the scheduled service;
synchronizing the scheduled service to the one or more service providers via a set of event streams, wherein the set of event streams detect and propagate real-time updates associated with the request; and
outputting the service information to the user interface.

16. The computer-program product of claim 15, wherein the one or more encoding models include a word embeddings (Word2Vec, GloVe) model, a one-hot encoding model, or a label encoding model.

17. The computer-program product of claim 15, wherein the ontology-based terminology mapping includes:
identifying a set of terminologies from the normalized data using transformer-based language models including BERT, BioBERT, or SciBERT; and
generating transformed data by mapping the identified set of terminologies to a predefined, standardized terminology using one or more machine-learning models including BERTMap, OntoAlign, DeepMap, TransE, ComplEx, or RotatE.

18. The computer-program product of claim 15, wherein the record linkage model includes a support vector machine (SVM), a pairwise logistic regression model, expectation-maximization (EM) algorithm, or a Siamese network model.

19. The computer-program product of claim 15, wherein an event stream of the set of event streams is a transactional event queue (TEQ) that comprises:
an event listener that detects real-time updates associated with the request; and
a message broker that delivers the detected real-time updates to the plurality of service providers.

20. The computer-program product of claim 15, wherein the set of operations further includes:
detecting, via an event stream of the set of event streams, an update within a datastore of the plurality of datastores, wherein the update is associated with at least one of: registering the user or scheduling the service; and
synchronizing, via the set of decoupled connectors, the update to the schema-agnostic datastore.
